(19) United States
(12) Patent Application Publication
(10) Pub. No.: US 2023/0165227 A1
(43) Pub. Date: Jun. 1, 2023

(54) GENETICALLY MODIFIED NON-HUMAN ANIMAL WITH HUMAN OR CHIMERIC CD276

(71) Applicant: Biocytogen Pharmaceuticals (Beijing) Co., Ltd., Beijing (CN)

(72) Inventors: Yuelei Shen, Beijing (CN); Yang Bai, Beijing (CN); Jiawei Yao, Beijing (CN); Yanan Guo, Beijing (CN); Lei Zhao, Beijing (CN)

(21) Appl. No.: 17/922,682

(22) PCT Filed: May 21, 2021
(86) PCT No.: PCT/CN2021/095273

§ 371 Date: Nov. 1, 2022

(30) Foreign Application Priority Data
May 21, 2020 (CN) 202010437339.3
Mar. 12, 2021 (CN) 202110267561.8

(51) Int. Cl. A01K 67/027; A61K 49/00; C07K 14/705; C07K 16/28

(52) U.S. Cl. CPC A01K 67/0278; A61K 49/0008; C07K 14/70532; C07K 16/2827; A01K 2227/105; A01K 2217/072; A01K 2267/0331; C07K 2319/02; C07K 2319/03

(57) ABSTRACT

The present disclosure relates to genetically modified non-human animals that express a human or chimeric (e.g., humanized) CD276, and methods of use thereof.

### CLAIM OF PRIORITY

This application is a National Phase Application under 35 U.S.C. § 371 and claims the benefit of International Patent Application No. PCT/CN2021/095273, filed on May 21, 2021, which claims the benefit of Chinese Patent Application App. No. 202010437339.3, filed on May 21, 2020 and Chinese Patent Application App. No. 202110267561.8, filed on Mar. 12, 2021.

The entire contents of the foregoing applications are incorporated herein by reference.

### TECHNICAL FIELD

This disclosure relates to genetically modified animal expressing human or chimeric (e.g., humanized) CD276, and methods of use thereof.

### BACKGROUND

The immune system has developed multiple mechanisms to prevent deleterious activation of immune cells. One such mechanism is the intricate balance between positive and negative co-stimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of humanized animal models that are suitable for human antibody screening and evaluation will significantly improve the efficiency of new drug development and reduce the cost for drug research and development.

### SUMMARY

This disclosure is related to an animal model with human CD276 or chimeric CD276. The animal model can express human CD276 or chimeric CD276 (e.g., humanized CD276) protein in its body. It can be used in the studies on the function of CD276 gene, and can be used in the screening and evaluation of anti-human CD276 antibodies. In addition, the animal models prepared by the methods described herein can be used in drug screening, pharmacodynamics studies, treatments for immune-related diseases (e.g., autoimmune diseases), and cancer therapy for human CD276 target sites; they can also be used to facilitate the development and design of new drugs, and save time and cost. In summary, this disclosure provides a powerful tool for studying the function of CD276 protein and a platform for screening cancer drugs.

In one aspect, the disclosure is related to a genetically-modified, non-human animal whose genome comprises at least one chromosome comprising a sequence encoding a human or chimeric CD276.

In some embodiments, the sequence encoding the human or chimeric CD276 is operably linked to an endogenous regulatory element at the endogenous CD276 gene locus in the at least one chromosome.

In some embodiments, the sequence encoding a human or chimeric CD276 comprises a sequence encoding an amino acid sequence that is at least 70%, 75%, 80%, 85%, 90%, 95%, 99%, or 100% identical to human CD276 (NP_001019907.1 (SEQ ID NO: 4)).

In some embodiments, the sequence encoding a human or chimeric CD276 comprises a sequence encoding an amino acid sequence that is at least 70%, 75%, 80%, 85%, 90%, 95%, 99%, or 100% identical to SEQ ID NO: 13.

In some embodiments, the sequence encoding a human or chimeric CD276 comprises a sequence that is at least 70%, 75%, 80%, 85%, 90%, 95%, 99%, or 100% identical to amino acids 34-417, 32-456, 30-459, or 26-459 of SEQ ID NO: 4.

In some embodiments, the animal is a mammal, e.g., a monkey, a rodent, or a mouse. In some embodiments, the animal is a mouse.

In some embodiments, the animal does not express endogenous CD276.

In some embodiments, the animal has one or more cells expressing human or chimeric CD276.

In some embodiments, the animal has one or more cells expressing human or chimeric CD276, and a human CD276 receptor can bind to the expressed human or chimeric CD276. In some embodiments, the animal has one or more cells expressing human or chimeric CD276, and an endogenous CD276 receptor can bind to the expressed human or chimeric CD276.

In one aspect, the disclosure is related to a genetically-modified, non-human animal. In some embodiments, the genome of the animal comprises a replacement of a sequence encoding a region of endogenous CD276 with a sequence encoding a corresponding region of human CD276 at an endogenous CD276 gene locus.

In some embodiments, the sequence encoding the corresponding region of human CD276 is operably linked to an endogenous regulatory element at the endogenous CD276 locus, and one or more cells of the animal expresses a chimeric CD276.

In some embodiments, the animal does not express endogenous CD276.

In some embodiments, the replaced sequence encodes all or a portion of the extracellular region (with or without signal peptide) of endogenous CD276.

In some embodiments, the animal has one or more cells expressing a chimeric CD276 having an extracellular region, a transmembrane region, and a cytoplasmic region, In some embodiments, the extracellular region comprises a sequence that is at least 50%, 60%, 70%, 80%, 90%, 95%, or 99% identical to the extracellular region of human CD276.

In some embodiments, the extracellular region of the chimeric CD276 has a sequence that has at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, or 430 contiguous amino acids that are identical to a contiguous sequence present in the extracellular region of human CD276.

In some embodiments, the sequence encoding a region of endogenous CD276 comprises exon 2, exon 3, exon 4, exon 5, exon 6, exon 7, and/or exon 8, or a part thereof, of the endogenous CD276 gene. In some embodiments, the animal is a mouse, and the sequence encoding a region of endogenous CD276 starts within exon 3 and ends within exon 4 of the endogenous mouse CD276 gene.

In some embodiments, the animal is heterozygous with respect to the replacement at the endogenous CD276 gene locus. In some embodiments, the animal is homozygous with respect to the replacement at the endogenous CD276 gene locus.

In one aspect, the disclosure is related to a method for making a genetically-modified, non-human animal, comprising: replacing in at least one cell of the animal, at an endogenous CD276 gene locus, a sequence encoding a region of an endogenous CD276 with a sequence encoding a corresponding region of human CD276.

In some embodiments, the sequence encoding the corresponding region of human CD276 comprises exon 2, exon 3, exon 4, exon 5, exon 6, exon 7, exon 8, exon 9, and/or exon 10, or a part thereof, of a human CD276 gene.

In some embodiments, the sequence encoding the corresponding region of human CD276 starts within exon 3 and ends within exon 6 of a human CD276 gene.

In some embodiments, the sequence encoding the corresponding region of human CD276 encodes amino acids 34-417 or 32-456 of SEQ ID NO: 4.

In some embodiments, the region of an endogenous CD276 is located within the extracellular region.

In some embodiments, the sequence encoding a region of endogenous CD276 comprises exon 2, exon 3, exon 4, exon 5, exon 6, exon 7, and/or exon 8, or a part thereof, of the endogenous CD276 gene. In some embodiments, the animal is a mouse, and the sequence encoding a region of an endogenous CD276 starts within exon 3 and ends within exon 4 of the endogenous mouse CD276 gene.

In one aspect, the disclosure is related to a non-human animal comprising at least one cell comprising a nucleotide sequence encoding a chimeric CD276 polypeptide. In some embodiments, the chimeric CD276 polypeptide comprises at least 50 contiguous amino acid residues that are identical to the corresponding contiguous amino acid sequence of a human CD276, In some embodiments, the animal expresses the chimeric CD276.

In some embodiments, the chimeric CD276 polypeptide has at least 50, at least 100, at least 150, at least 200, or at least 250 contiguous amino acid residues that are identical to the corresponding contiguous amino acid sequence of a human CD276 extracellular region.

In some embodiments, the chimeric CD276 polypeptide comprises a sequence that is at least 90%, 95%, or 99% identical to amino acids 34-417, 32-456, 30-459, or 26-459 of SEQ ID NO: 4.

In some embodiments, the nucleotide sequence is operably linked to an endogenous CD276 regulatory element of the animal.

In some embodiments, the chimeric CD276 polypeptide comprises an endogenous CD276 transmembrane region and/or an endogenous CD276 cytoplasmic region.

In some embodiments, the nucleotide sequence is integrated to an endogenous CD276 gene locus of the animal.

In some embodiments, the chimeric CD276 has at least one mouse CD276 activity and/or at least one human CD276 activity.

In one aspect, the disclosure is related to a method of making a genetically-modified non-human animal cell that expresses a chimeric CD276, the method comprising: replacing at an endogenous CD276 gene locus, a nucleotide sequence encoding a region of endogenous CD276 with a nucleotide sequence encoding a corresponding region of human CD276, thereby generating a genetically-modified non-human animal cell that includes a nucleotide sequence that encodes the chimeric CD276. In some embodiments, the non-human animal cell expresses the chimeric CD276.

In some embodiments, the animal is a mammal, e.g., a monkey, a rodent, or a mouse.

In some embodiments, the chimeric CD276 comprises: an extracellular region of human CD276, optionally comprising an endogenous signal peptide sequence; and a transmembrane and/or a cytoplasmic region of endogenous CD276.

In some embodiments, the nucleotide sequence encoding the chimeric CD276 is operably linked to an endogenous CD276 regulatory region, e.g., promoter.

In some embodiments, the animal further comprises a sequence encoding an additional human or chimeric protein. In some embodiments, the additional human or chimeric protein is programmed cell death protein 1 (PD-1), IL4, Colony Stimulating Factor 1 (CSF1), IL34, C—C Motif Chemokine Receptor 2 (CCR2), CD40, C—X—C Motif Chemokine Receptor 4 (CXCR4), Vascular Endothelial Growth Factor (VEGF), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immuno receptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (OX40).

In some embodiments, the additional human or chimeric protein is PD-1, and the animal expresses the human or chimeric PD-1.

In one aspect, the disclosure is related to a method of determining effectiveness of an anti-CD276 antibody for the treatment of cancer, comprising: administering the anti-CD276 antibody to the animal as described herein, in some embodiments, the animal has a cancer; and determining the inhibitory effects of the anti-CD276 antibody to the cancer.

In some embodiments, the cancer comprises one or more cells that express CD276.

In some embodiments, the cancer comprises one or more cancer cells that are injected into the animal.

In some embodiments, determining the inhibitory effects of the anti-CD276 antibody to the cancer involves measuring the tumor volume in the animal.

In some embodiments, the cancer is liver cancer, pancreatic cancer, prostate cancer, osteosarcoma, breast cancer, colorectal cancer, stomach cancer, ovarian cancer, endometrial cancer, oral squamous cell carcinoma, cervical cancer, non-small cell lung cancer (NSCLC), bladder cancer, renal cancer, brain cancer, or melanoma.

In one aspect, the disclosure is related to a method of determining effectiveness of an anti-CD276 antibody and an additional therapeutic agent for the treatment of cancer, comprising administering the anti-CD276 antibody and the additional therapeutic agent to the animal as described herein, in some embodiments, the animal has a cancer; and determining the inhibitory effects on the cancer.

In some embodiments, the animal further comprises a sequence encoding a human or chimeric programmed cell death protein 1 (PD-1).

In some embodiments, the animal further comprises a sequence encoding a human or chimeric programmed death-ligand 1 (PD-L1).

In some embodiments, the additional therapeutic agent is an anti-PD-1 antibody or an anti-PD-L1 antibody.

In some embodiments, the cancer comprises one or more cancer cells that express CD276, PD-L1, or PD-L2.

In some embodiments, the cancer is caused by injection of one or more cancer cells into the animal.

In some embodiments, determining the inhibitory effects of the treatment involves measuring the tumor volume in the animal.

In some embodiments, the animal has liver cancer, pancreatic cancer, prostate cancer, osteosarcoma, breast cancer, colorectal cancer, stomach cancer, ovarian cancer, endometrial cancer, oral squamous cell carcinoma, cervical cancer, non-small cell lung cancer (NSCLC), bladder cancer, renal cancer, brain cancer, head and neck cancer, or melanoma.

In one aspect, the disclosure is related to a protein comprising an amino acid sequence, in some embodiments, the amino acid sequence is one of the following:
(a) an amino acid sequence set forth in SEQ ID NO: 2, 4, or 13;
(b) an amino acid sequence that is at least 90% identical to SEQ ID NO: 2, 4, or 13;
(c) an amino acid sequence that is at least 91%, 92%, 93%, 94%, 95%, 96%, 97%, 98%, or 99% identical to SEQ ID NO: 2, 4, or 13;
(d) an amino acid sequence that is different from the amino acid sequence set forth in SEQ ID NO: 2, 4, or 13 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2, 4, or 13.

In one aspect, the disclosure is related to a nucleic acid comprising a nucleotide sequence, in some embodiments, the nucleotide sequence is one of the following:
(a) a sequence that encodes the protein as described herein;
(b) SEQ ID NO: 1, 3, 5, 6, 7, 8, 9, 10, 11, or 12;
(c) a sequence that is at least 90% identical to SEQ ID NO: 1, 3, 5, 6, 7, 8, 9, 10, 11, or 12; and
(d) a sequence that is at least 91%, 92%, 93%, 94%, 95%, 96%, 97%, 98%, or 99% identical to SEQ ID NO: 1, 3, 5, 6, 7, 8, 9, 10, 11, or 12.

In one aspect, the disclosure is related to a cell comprising the protein and/or the nucleic acid as described herein. In one aspect, the disclosure is related to an animal comprising the protein and/or the nucleic acid as described herein.

In some embodiments, the mice described in the present disclosure can be bred with the mice containing other human or chimeric genes (e.g., chimeric PD-1, chimeric PD-L1, chimeric CTLA-4, or other immunomodulatory factors), so as to obtain a mouse expressing two or more human or chimeric proteins. The mice can also, e.g., be used for screening antibodies in the case of a combined use of drugs, as well as evaluating the efficacy of the combination therapy.

The disclosure also relates to non-human mammal generated through the methods as described herein. In some embodiments, the genome thereof contains human gene(s). In some embodiments, the non-human mammal is a rodent. In some embodiments, the non-human mammal is a mouse. In some embodiments, the non-human mammal expresses a protein encoded by a humanized CD276 gene.

The disclosure also relates to an offspring of the non-human mammal.

In another aspect, the disclosure relates to a tumor bearing non-human mammal model, characterized in that the non-human mammal model is obtained through the methods as described herein. In some embodiments, the non-human mammal is a rodent. In some embodiments, the non-human mammal is a mouse.

The disclosure also relates to a cell (e.g., stem cell or embryonic stem cell) or cell line, or a primary cell culture thereof derived from the non-human mammal or an offspring thereof, or the tumor bearing non-human mammal.

The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to a CD276 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the CD276 gene function, human CD276 antibodies, the drugs or efficacies for human CD276 targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic diagram showing mouse CD276 gene locus.

FIG. 1B is a schematic diagram showing human CD276 gene locus.

FIG. 2 is a schematic diagram showing humanized CD276 gene locus.

FIG. 3 is a schematic diagram showing a CD276 gene targeting strategy.

FIG. 4 shows Southern Blot results of cells after recombination using the 5′ Probe, 3′ Probe, Neo Probe. D01, D02, D03, D04, D05, D06, D07, D08, and D09 are clone numbers. WT is a wild-type control.

FIG. 5 is a schematic diagram showing the FRT recombination process.

FIG. 6A shows PCR identification results of F1 generation mice by primers WT-F and WT-R. F1-1, F1-2, and F1-3 are mouse numbers. M is a marker. PC is a positive control (Neo cassette removed). WT is a wild-type control. H₂O is a water control.

FIG. 6B shows PCR identification results of F1 generation mice by primers WT-F and Mut-R. F1-1, F1-2, and F1-3 are mouse numbers. M is a marker. PC is a positive control (Neo cassette removed). WT is a wild-type control. H₂O is a water control.

FIG. 6C shows PCR identification results of F1 generation mice by primers Frt-F and Frt-R. F1-1, F1-2, and F1-3 are mouse numbers. M is a marker. PC is a positive control (Neo cassette removed). WT is a wild-type control. H₂O is a water control.

FIG. 7 shows RT-PCR identification results of wild-type C57BL/6 mice (+/+) or CD276 gene humanized homozygous mice (H/H) to detect expression of mouse CD276 (mB7-H3), human CD276 (hB7-H3), and human GAPDH. M is a marker. H₂O is a water control.

FIG. 8 shows the average body weight of CD276 gene humanized homozygous mice that were xenografted with MC38-hCD276 cells, and then treated with anti-human CD276 antibody Enoblituzumab at 10 mg/kg (G2). 10 mg/kg human IgG (hIgG) was administered as a control (GI).

FIG. 9 shows the average body weight change of CD276 gene humanized homozygous mice that were xenografted with MC38-hCD276 cells, and then treated with anti-human CD276 antibody Enoblituzumab at 10 mg/kg (G2). 10 mg/kg human IgG (hIgG) was administered as a control (G1).

FIG. 10 shows the tumor volume of CD276 gene humanized homozygous mice that were xenografted with MC38-hCD276 cells, and then treated with anti-human CD276 antibody Enoblituzumab at 10 mg/kg (G2). 10 mg/kg human IgG (hIgG) was administered as a control (GI).

FIG. 11 shows the alignment between human CD276 amino acid sequence (NP_001019907.1; SEQ ID NO: 4) and mouse CD276 amino acid sequence (NP_598744.1; SEQ ID NO: 2).

FIG. 12 shows the average body weight of CD276 gene humanized homozygous mice that were xenografted with MC38-hCD276 cells, and then treated with anti-mouse PD-1 antibody (G2), anti-human CD276 antibody Enoblituzumab (G3), or a combination of anti-mouse PD-1 antibody and Enoblituzumab (G4). Human IgG (hIgG) was administered as a control (G1).

FIG. 13 shows the average body weight change of CD276 gene humanized homozygous mice that were xenografted with MC38-hCD276 cells, and then treated with anti-mouse PD-1 antibody (G2), anti-human CD276 antibody Enoblituzumab (G3), or a combination of anti-mouse PD-1 antibody and Enoblituzumab (G4). Human IgG (hIgG) was administered as a control (G1).

FIG. 14 shows the tumor volume of CD276 gene humanized homozygous mice that were xenografted with MC38-hCD276 cells, and then treated with anti-mouse PD-1 antibody (G2), anti-human CD276 antibody Enoblituzumab (G3), or a combination of anti-mouse PD-1 antibody and Enoblituzumab (G4). Human IgG (hIgG) was administered as a control (G1).

FIG. 15 shows percentages of leukocyte subtypes in the spleen of C57BL/6 wild-type mice and CD276 gene humanized homozygous mice (B7-H3), as determined by flow cytometry.

FIG. 16 shows percentages of T cell subtypes in the spleen of C57BL/6 wild-type mice and CD276 gene humanized homozygous mice (B7-H3), as determined by flow cytometry.

FIG. 17 shows percentages of leukocyte subtypes in the lymph nodes of C57BL/6 wild-type mice and CD276 gene humanized homozygous mice (B7-H3), as determined by flow cytometry.

FIG. 18 shows percentages of T cell subtypes in the lymph nodes of C57BL/6 wild-type mice and CD276 gene humanized homozygous mice (B7-H3), as determined by flow cytometry.

FIG. 19 shows percentages of leukocyte subtypes in the peripheral blood of C57BL/6 wild-type mice and CD276 gene humanized homozygous mice (B7-H3), as determined by flow cytometry.

FIG. 20 shows percentages of T cell subtypes in the peripheral blood of C57BL/6 wild-type mice and CD276 gene humanized homozygous mice (B7-H3), as determined by flow cytometry.

FIG. 21 shows the alignment between human CD276 amino acid sequence (NP_001019907.1; SEQ ID NO: 4) and mouse CD276 amino acid sequence (NP_877976.1; SEQ ID NO: 35).

### DETAILED DESCRIPTION

This disclosure relates to transgenic non-human animal with human or chimeric (e.g., humanized) CD276, and methods of use thereof.

The B7 family consists of structurally related, cell-surface protein ligands, which bind to receptors on lymphocytes that regulate immune responses. Activation of T and B lymphocytes is initiated by engagement of cell-surface, antigen-specific T-cell receptors or B-cell receptors, but additional signals delivered simultaneously by B7 ligands determine the ultimate immune response. These “co-stimulatory” or “co-inhibitory” signals are delivered by B7 ligands through the CD28 family of receptors on lymphocytes. Interaction of B7-family members with co-stimulatory receptors augments immune responses, and interaction with co-inhibitory receptors attenuates immune responses. There are currently seven known members of the family: B7.1 (CD80), B7.2 (CD86), inducible co-stimulator ligand (ICOS-L), programmed death-1 ligand (PD-L1), programmed death-2 ligand (PD-L2), B7-H3 (CD276), and B7-H4. They are all transmembrane or glycosylphosphatidylinositol (GPI)-linked proteins characterized by extracellular IgV and IgC domains related to the variable and constant domains of immunoglobulins. The IgV and IgC domains of B7-family members are each encoded by single exons, with additional exons encoding leader sequences, transmembrane and cytoplasmic domains. B7-H3 (CD276) is unique in that the major human form contains two extracellular tandem IgV-IgC domains.

Members of the B7 family are expressed in antigen-presenting cells, including dendritic cells, macrophages, and B cells. Unlike B7.1, which is only expressed in lymphocytes, CD276 is also expressed in a variety of normal tissues, but the expression levels are low. Expression of CD276 is regulated by a variety of cytokines, for example, IFN-γ can up-regulate the expression of CD276, and IL4 can down-regulate its expression. The binding of B7 family members to different receptors can have a co-stimulatory or co-inhibitory effect. For example, binding of B7 and CD28 promotes co-stimulation, whereas binding of B7 and CTLA4 promotes co-inhibition. Early studies discovered that CD276 promotes the positive regulation of T cells, but subsequent studies found that CD276 plays a co-inhibitory effect in T cell immunity. Thus, CD276 has dual immune effects of co-stimulation and co-inhibition.

In non-malignant tissues, B7-H3 (CD276) has a predominantly inhibitory role in adaptive immunity, suppressing T cell activation and proliferation. In malignant tissues, B7-H3 is an immune checkpoint molecule that inhibits tumor antigen-specific immune responses. B7-H3 also possesses non-immunological pro-tumorigenic functions such as promoting migration, invasion, angiogenesis, chemoresistance, epithelial-to-mesenchymal transition, and affecting tumor cell metabolism. Thus, B7-H3 (CD276) antibodies can be potentially useful as cancer therapies.

Experimental animal models are an indispensable research tool for studying the effects of these antibodies (e.g., CD276 antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in details in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullis et al U.S. Pat. No. 4,683,195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N.Y., 1986); each of which is incorporated herein by reference in its entirety.

### CD276

CD276 (Cluster of Differentiation 276; also known as B7 Homolog 3 (B7-H3), B7RP-2, or 4Ig-B7-H3) is a type I transmembrane protein encoded by chromosome 9 in mice and chromosome 15 in humans. The extracellular domain is composed of a single pair of immunoglobulin variable domain and immunoglobulin constant domain in mice (2Ig-B7-H3 isoform) and two identical pairs in human (4Ig-B7-H3 isoform) due to exon duplication. The intracellular tail of B7-H3 is short and has no known signaling motif B7-H3 was first described in humans and then in mice, but is universally expressed among species. A soluble form, cleaved from the surface of activated T cell, monocyte, or DCs by a matrix metallopeptidase MMP or produced through alternative splicing of the intron, is also detectable in human sera. Soluble CD276 can activate the NF-κB signaling pathway to enhance the invasion and metastasis of pancreatic cancer cells. In addition, the level of soluble CD276 in the pleural effusion of patients with non-small cell lung cancer is significantly higher than that of healthy people. Thus, CD276 can be used as a diagnostic and prognostic indicator for related tumors.

B7-H3 is expressed on many tissues and cell types. At the mRNA level, it is ubiquitously found in non-lymphoid and lymphoid organs as liver, heart, prostate, spleen and thymus. Despite broad mRNA expression, protein expression is limited at steady state, suggesting the presence of an important post-transcriptional control mechanism. B7-H3 is constitutively found on non-immune resting fibroblasts, endothelial cells (EC), osteoblasts, and amniotic fluid stem cells. Moreover, B7-H3 expression is induced on immune cells, specifically antigen-presenting cells. In particular, coculture with regulatory T cells (Treg), IFN-7, lipopolysaccharide (LPS), or anti-CD40 in vitro stimulation all induce the expression of B7-H3 on dendritic cells (DCs). Monocytes and monocytes-derived DCs upregulate B7-H3 after LPS stimulation or cytokine-induced differentiation respectively. Additionally, B7-H3 is also detected on natural killer (NK) cells, B cells, and a minor population of T cells following PMA/ionomycin stimulation.

The B7-H3 pathway has a dual role in contributing to the regulation of innate immune responses. One study found that neuroblastoma cells express B7-H3 on their cell surface, which protect them from NK cell-mediated lysis. Another group argues that B7-H3 co-stimulates innate immunity by augmenting pro-inflammatory cytokines release from LPS-stimulated monocytes/macrophages, in both a Toll-like receptor 4- and 2-dependent manner.

A larger body of literature suggests that B7-H3 plays an important role in T cell-mediated adaptive immunity, although the nature of its signaling remains controversial. A co-stimulatory role of B7-H3 on human T cells was initially reported in vitro. Murine studies showing B7-H3 worsens experimental autoimmune encephalomyelitis (EAE), arthritis, bacterial meningitis and chronic allograft rejection supported this claim. However, subsequent studies have mostly shown that B7-H3 acts as a T cell co-inhibitor. B7-H3 inhibits polyclonal or allogeneic CD4+ and CD8+ T cell activation, proliferation and effector cytokine production (IFN-γ and IL-2) in mice and humans. This negative regulation of T cells is associated with diminished NFAT, NF-κB and AP-1 transcriptional factor activity. Independent studies utilizing either protein blockade or gene-knockout mice have reported that B7-H3 ameliorates graft-versus-host-disease, prolongs cardiac allograft survival, reduces airway hypersensitivity, and delays EAE onset, especially by down-regulating Th1 responses. These examples lend more credence to the co-inhibitory nature of B7-H3.

The receptor(s) for B7-H3 has yet to be discovered. Nevertheless, the crystal structure of mouse B7-H3 reveals that its receptor engagement on T cells involves the particular segment connecting F and G strands (the FG loop) of the immunoglobulin variable domain of B7-H3. Moreover, B7-H3 crystallizes as a glycosylated monomer but also undergoes an unusual dimerization in vitro. Altogether, the nature of the receptor(s), differences in cellular context, and various disease models certainly account for the discrepancies in the function of the B7-H3 pathway in regulating both innate and adaptive immunity during homeostasis and inflammation.

Beyond the immune system, the B7-H3 pathway has a non-immunological role in promoting osteoblastic differentiation and bone mineralization in mice, ensuring normal bone formation. Indeed, B7-H3 knockout mice had reduced bone mineral density and were more susceptible to bone fractures compared to wild-type mice. Furthermore, similar to other immune checkpoints of the B7-CD28 pathways, B7-H3 is also expressed in human cancers and participates in tumorigenesis through modulation of both immune and non-immune related pathways.

A detailed description of CD276 and its function can be found, e.g., in Picarda, E. et al., “Molecular pathways: targeting B7-H3 (CD276) for human cancer immunotherapy.” Clinical Cancer Research 22.14 (2016): 3425-3431; Collins, M. et al., “The B7 family of immune-regulatory ligands.” Genome Biology 6.6 (2005): 1-7; Castellanos, J. R. et al., “B7-H3 role in the immune landscape of cancer.” American Journal of Clinical and Experimental Immunology 6.4 (2017): 66; and Yang, S. et al., “B7-H3, a checkpoint molecule, as a target for cancer immunotherapy.” International Journal of Biological Sciences 16.11 (2020): 1767; each of which is incorporated by reference in its entirety.

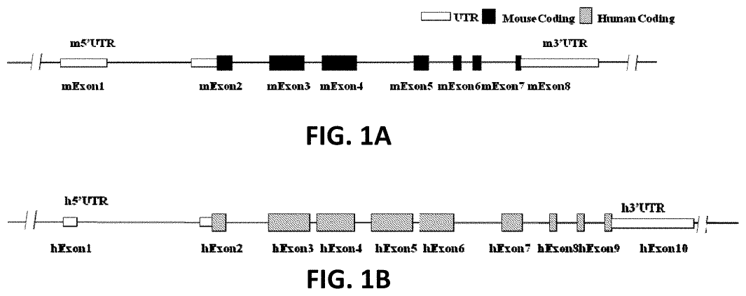

In human genomes, CD276 gene (Gene ID: 80381) locus has ten exons, exon 1, exon 2, exon 3, exon 4, exon 5, exon 6, exon 7, exon 8, exon 9, and exon 10 (FIG. 1B). The CD276 protein also has an extracellular region, a transmembrane region, and a cytoplasmic region, and the signal peptide is located at the extracellular region of CD276. The nucleotide sequence for human CD276 mRNA is NM_001024736.2 (SEQ ID NO: 3), and the amino acid sequence for human CD276 is NP_001019907.1 (SEQ ID NO: 4). The location for each exon and each region in human CD276 nucleotide sequence and amino acid sequence is listed below:

TABLE 1

| Human CD276 | NM_001024736.2 | NP_001019907.1 |
|---|---|---|
| (approximate | 3295 bp | 534 aa |
| location) | SEQ ID NO: 3 | SEQ ID NO: 4 |
| Exon 1 | 1-78 | — |
| Exon 2 | 79-211 | 1-26 |
| Exon 3 | 212-550 | 27-139 |
| Exon 4 | 551-865 | 140-244 |
| Exon 5 | 866-1204 | 245-357 |
| Exon 6 | 1205-1501 | 358-456 |
| Exon 7 | 1502-1636 | 457-501 |
| Exon 8 | 1637-1678 | 502-515 |
| Exon 9 | 1679-1714 | 516-527 |
| Exon 10 | 1715-3295 | 528-534 |
| Extracellular | 217-1530 | 29-466 |
| Transmembrane | 1531-1593 | 467-487 |
| Cytoplasmic | 1594-1734 | 488-534 |
| Signal peptide | 133-216 | 1-28 |
| Replaced region | 232-1383 | 34-417 |
| in Example | | |

The human CD276 gene (Gene ID: 80381) is located in Chromosome 15 of the human genome, which is located from 73683966 to 73714518 of NC_000015.10 (GRCh38.p13 (GCF_000001405.39)). The 5′-UTR is from 73,684,383 to 73699639, exon 1 is from 73,684,383 to 73,684,460, the first intron is from 73,684,461 to 73,699,585, exon 2 is from 73,699,586 to 73,699,718, the second intron is from 73,699,719 to 73,702,254, exon 3 is from 73,702,255 to 73,702,593, the third intron is from 73,702,594 to 73,702,771, exon 4 is from 73,702,772 to 73,703,086, the forth intron is from 73,703,087 to 73,703,658, exon 5 is from 73,703,659 to 73,703,997, the fifth intron is from 73,703,998 to 73,704,175, exon 6 is from 73,704,176 to 73,704,472, the sixth intron is from 73,704,473 to 73,708,338, exon 7 is from 73,708,339 to 73,708,473, the seven intron is from 73,708,474 to 73,709,647, exon 8 is from 73,709,648 to 73,709,689, the eighth intron is from 73,709,690 to 73,711,134, exon 9 is from 73,711,135 to 73,711,170, the nine intron is from 73,711,171 to 73,712,933, exon 10 is from 73,712,934 to 73,714,514, and the 3′-UTR is from 73712957 to 73714514, based on transcript NM_001024736.2. All relevant information for human CD276 locus can be found in the NCBI website with Gene ID: 80381, which is incorporated by reference herein in its entirety.

According to the UniProt Database (UniProt ID: Q5ZPR3), the extracellular region (not including signal peptide) of human CD276 corresponds to amino acids 29-466 of SEQ ID NO: 4; the transmembrane region of human CD276 corresponds to amino acids 467-487 of SEQ ID NO: 4; the cytoplasmic region of human CD276 corresponds to amino acids 488-534 of SEQ ID NO: 4; the signal peptide of human CD276 corresponds to amino acids 1-28 of SEQ ID NO: 4. Specifically, there are four Ig-like domains within the extracellular region of human CD276. For N-terminus to C-terminus of human CD276 extracellular region, the Ig-like V-type 1 domain corresponds to amino acids 29-139 of SEQ ID NO: 4; the Ig-like C2-type 1 domain corresponds to amino acids 145-238 of SEQ ID NO: 4; the Ig-like V-type 2 domain corresponds to amino acids 243-357 of SEQ ID NO: 4; and the Ig-like C2-type 2 domain corresponds to amino acids 363-456 of SEQ ID NO: 4.

In mice, CD276 gene locus has eight exons, exon 1, exon 2, exon 3, exon 4, exon 5, exon 6, exon 7, and exon 8 (FIG. 1A). The mouse CD276 protein also has an extracellular region, a transmembrane region, and a cytoplasmic region, and the signal peptide is located at the extracellular region of CD276. The nucleotide sequence for mouse CD276 cDNA is NM_133983.4 (SEQ ID NO: 1), the amino acid sequence for mouse CD276 is NP_598744.1 (SEQ ID NO: 2). The location for each exon and each region in the mouse CD276 nucleotide sequence and amino acid sequence is listed below:

TABLE 2

| Mouse CD276 | NM_133983.4 | NP_598744.1 |
|---|---|---|
| (approximate | 3197 bp | 316 aa |
| location) | SEQ ID NO: 1 | SEQ ID NO: 2 |
| Exon 1 | 1-12 | — |
| Exon 2 | 13-286 | 1-26 |
| Exon 3 | 287-625 | 27-139 |
| Exon 4 | 626-922 | 140-238 |
| Exon 5 | 923-1057 | 239-283 |
| Exon 6 | 1058-1099 | 284-297 |
| Exon 7 | 1100-1135 | 298-309 |
| Exon 8 | 1136-3197 | 310-316 |
| Extracellular | 292-951 | 29-248 |
| Transmembrane | 952-1014 | 249-269 |
| Cytoplasmic | 1015-1155 | 270-316 |
| Signal peptide | 208-291 | 1-28 |
| Replaced region | 307-804 | 34-199 |
| in Example | | |

The mouse CD276 gene (Gene ID: 102657) is located in Chromosome 9 of the mouse genome, which is located from 58524300 to 58555033 of NC_000075.6 (GRCm38.p6 (GCF_000001635.26)). The 5′-UTR is from 58,555,437 to 58540746, exon 1 is from 58,555,437 to 58,555,022, the first intron is from 58,555,021 to 58,540,941, exon 2 is from 58,540,940 to 58,540,667, the second intron is from 58,540,666 to 58,537,608, exon 3 is from 58,537,607 to 58,537,269, the third intron is from 58,537,268 to 58,535,753, exon 4 is from 58,535,752 to 58,535,456, the forth intron is from 58,535,455 to 58,530,731, exon 5 is from 58,530,730 to 58,530,596, the fifth intron is from 58,530,595 to 58,528,494, exon 6 is from 58,528,493 to 58,528,452, the sixth intron is from 58,528,451 to 58,527,555, exon 7 is from 58,527,554 to 58,527,519, the seven intron is from 58,527,518 to 58,526,362, exon 8 is from 58,527,518 to 58,526,361, and the 3′-UTR is from 58524298 to 58526338, based on transcript NM_133983.4. All relevant information for mouse CD276 locus can be found in the NCBI website with Gene ID: 102657, which is incorporated by reference herein in its entirety.

According to the UniProt Database (UniProt ID: Q8VE98), the extracellular region (not including signal peptide) of mouse CD276 corresponds to amino acids 29-248 of SEQ ID NO: 2; the transmembrane region of mouse CD276 corresponds to amino acids 249-269 of SEQ ID NO: 2; the cytoplasmic region of mouse CD276 corresponds to amino acids 270-316 of SEQ ID NO: 2; the signal peptide of mouse CD276 corresponds to amino acids 1-28 of SEQ ID NO: 2. Specifically, there are two Ig-like domains within the extracellular region of mouse CD276. For N-terminus to C-terminus of mouse CD276 extracellular region, the Ig-like V-type domain corresponds to amino acids 29-139 of SEQ ID NO: 2; and the Ig-like C2-type domain corresponds to amino acids 145-238 of SEQ ID NO: 2.

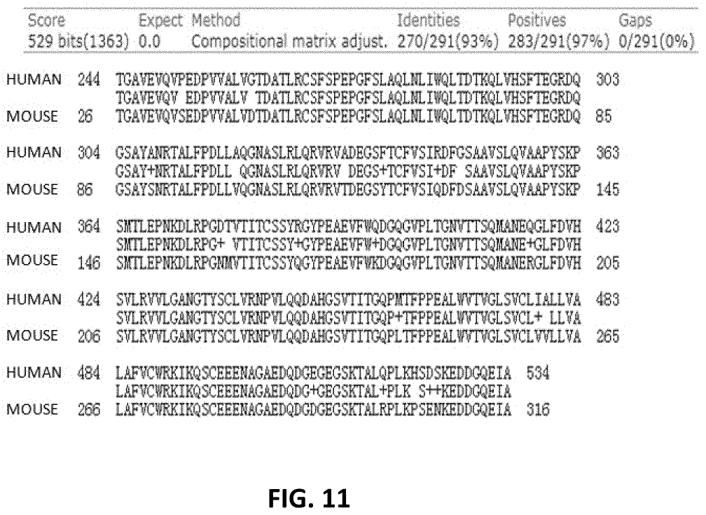

FIG. 11 shows the alignment between human CD276 amino acid sequence (NP_001019907.1; SEQ ID NO: 4) and mouse CD276 amino acid sequence (NP_598744.1; SEQ ID NO: 2). Thus, the corresponding amino acid residue or region between human and mouse CD276 can be found in FIG. 11.

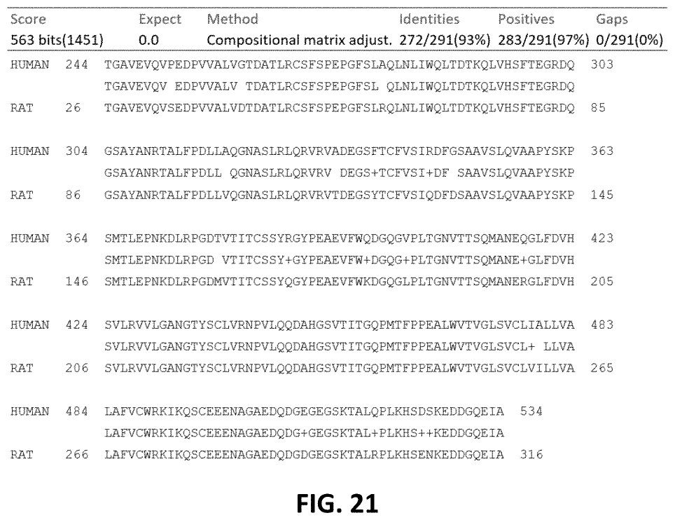

CD276 genes, proteins, and locus of the other species are also known in the art. For example, the gene ID for CD276 in Rattus norvegicus (rat) is 315716, the gene ID for CD276 in Macaca mulatta (Rhesus monkey) is 699877, the gene ID for CD276 in Canis lupus familiaris (dog) is 487638, and the gene ID for CD276 in Equus caballus (horse) is 100052046. The relevant information for these genes (e.g., intron sequences, exon sequences, amino acid residues of these proteins) can be found, e.g., in NCBI database, which is incorporated by reference herein in its entirety. FIG. 21 shows the alignment between human CD276 amino acid sequence (NP_001019907.1; SEQ ID NO: 4) and rodent CD276 amino acid sequence (NP_877976.1; SEQ ID NO: 35). Thus, the corresponding amino acid residue or region between human and rodent CD276 can be found in FIG. 21.

The present disclosure provides human or chimeric (e.g., humanized) CD276 nucleotide sequence and/or amino acid sequences. In some embodiments, the entire sequence of mouse exon 1, exon 2, exon 3, exon 4, exon 5, exon 6, exon 7, exon 8, Ig-like V-type domain, Ig-like C2-type domain, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, 600, 700, 800, 900, 1000, 1100, 1200, or 1300 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, or 430 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, signal peptide, extracellular region, transmembrane region, or cytoplasmic region of mouse CD276 gene; or exon 1, exon 2, exon 3, exon 4, exon 5, exon 6, exon 7, exon 8, exon 9, exon 10, signal peptide, extracellular region, transmembrane region, or cytoplasmic region of human CD276 gene. In some embodiments, a region, a portion, or the entire sequence of mouse exon 1, exon 2, exon 3, exon 4, exon 5, exon 6, exon 7, and/or exon 8 (e.g., a portion of exon 3 and a portion of exon 4) are replaced by human exon 1, exon 2, exon 3, exon 4, exon 5, exon 6, exon 7, exon 8, exon 9, and/or exon 10 (e.g., a portion of exon 3, exons 4-5, and a portion of exon 6) sequence.

In some embodiments, the genetically-modified non-human animal described herein comprises a sequence encoding a humanized CD276 protein. In some embodiments, the humanized CD276 protein comprises a humanized extracellular region. In some embodiments, the humanized CD276 protein comprises an endogenous signal peptide. In some embodiments, the humanized CD276 protein comprises an endogenous transmembrane region. In some embodiments, the humanized CD276 protein comprises an endogenous cytoplasmic region. In some embodiments, the humanized CD276 protein comprises humanized Ig-like domains (e.g., humanized Ig-like V-type and/or C-type domains).

In some embodiments, the genetically-modified non-human animal described herein comprises a humanized CD276 gene. In some embodiments, the humanized CD276 gene comprises 10 exons. In some embodiments, the humanized CD276 gene comprises humanized exon 1, humanized exon 2, humanized exon 3, humanized exon 4, humanized exon 5, humanized exon 6, humanized exon 7, humanized exon 8, humanized exon 9, and/or humanized exon 10.

In some embodiments, the present disclosure also provides a chimeric (e.g., humanized) CD276 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD276 mRNA sequence (e.g., SEQ ID NO: 1), mouse CD276 amino acid sequence (e.g., SEQ ID NO: 2), or a portion thereof (e.g., exons 1-2, a portion of exon 3, a portion of exon 4, and exons 5-8 of mouse CD276 gene); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD276 mRNA sequence (e.g., SEQ ID NO: 3), human CD276 amino acid sequence (e.g., SEQ ID NO: 4), or a portion thereof (e.g., a portion of exon 3, exons 4-5, and a portion of exon 6 of human CD276 gene).

In some embodiments, the sequence encoding amino acids 34-199 of mouse CD276 (SEQ ID NO: 2) is replaced. In some embodiments, the sequence is replaced by a sequence encoding a corresponding region of human CD276 (e.g., amino acids 34-417 of human CD276 (SEQ ID NO: 4)).

In some embodiments, the sequence encoding amino acids 32-238 of mouse CD276 (SEQ ID NO: 2) is replaced. In some embodiments, the sequence is replaced by a sequence encoding a corresponding region of human CD276 (e.g., amino acids 32-456 of human CD276 (SEQ ID NO: 4)).

In some embodiments, the sequence encoding amino acids 30-241 of mouse CD276 (SEQ ID NO: 2) is replaced. In some embodiments, the sequence is replaced by a sequence encoding a corresponding region of human CD276 (e.g., amino acids 30-459 of human CD276 (SEQ ID NO: 4)).

In some embodiments, the sequence encoding amino acids 26-241 of mouse CD276 (SEQ ID NO: 2) is replaced. In some embodiments, the sequence is replaced by a sequence encoding a corresponding region of human CD276 (e.g., amino acids 26-459 of human CD276 (SEQ ID NO: 4)).

In some embodiments, the sequence encoding the entirety or a portion of the extracellular region (with or without the signal peptide) of mouse CD276 (SEQ ID NO: 2) is replaced or inactivated. In some embodiments, the sequence is replaced by a sequence encoding the entirety or a portion of the corresponding region of human CD276 (SEQ ID NO: 4). In some embodiments, the corresponding region of human CD276 comprises the entirety or a portion of the extracellular region (with or without the signal peptide) of human CD276. In some embodiments, the sequence encoding amino acids 29-248 of mouse CD276 (SEQ ID NO: 2) is replaced. In some embodiments, the sequence is replaced by a sequence encoding a corresponding region of human CD276 (e.g., amino acids 29-466 of human CD276 (SEQ ID NO: 4)). In some embodiments, the sequence encoding amino acids 1-248 of mouse CD276 (SEQ ID NO: 2) is replaced. In some embodiments, the sequence is replaced by a sequence encoding a corresponding region of human CD276 (e.g., amino acids 1-466 of human CD276 (SEQ ID NO: 4)). In some embodiments, the sequence encoding the corresponding region of human CD276 does not include at least 1, at least 2, at least 3, at least 4, at least 5, at least 6, at least 7, at least 8, at least 9, or at least 10 amino acids at the N-terminus and/or C-terminus of the extracellular region of human CD276.

In some embodiments, the sequence encoding the extracellular Ig-like V-type domain and Ig-like C2-type domain of mouse CD276 (SEQ ID NO: 2) is replaced or inactivated. In some embodiments, the sequence is replaced by a sequence encoding the extracellular Ig-like V-type 1 domain, Ig-like C2-type 1 domain, Ig-like V-type 2 domain, and Ig-like C2-type 2 domain of human CD276 (SEQ ID NO: 4). In some embodiments, the sequence encoding amino acids 29-238 of mouse CD276 (SEQ ID NO: 2) is replaced. In some embodiments, the sequence is replaced by a sequence encoding amino acids 29-456 of human CD276 (SEQ ID NO: 4).

While not intending to be bound by any theory, the correspondence between the Ig-like domains of mouse CD276 with one or both duplicate of the Ig-like domains of human CD276 can be established, due to exon duplication in human CD276 gene. In some embodiments, the sequence encoding the Ig-like domains of mouse CD276 (SEQ ID NO: 2) is replaced with a sequence encoding either one (e.g., the Ig-like V-type 1 domain plus Ig-like C2-type 1 domain, or the Ig-like V-type 2 domain plus Ig-like C2-type 2 domain) or both duplicates (e.g., the extracellular Ig-like V-type 1 domain, Ig-like C2-type 1 domain, Ig-like V-type 2 domain, and Ig-like C2-type 2 domain) of the Ig-like domains of human CD276 (SEQ ID NO: 4). In some embodiments, the genetically modified non-human animal (e.g., a mouse) described herein includes one or more (e.g., 1, 2, 3, or 4) humanized Ig-like domains (e.g., Ig-like V-type domains and/or Ig-like C2 type domains).

In some embodiments, the nucleic acids as described herein are operably linked to a promotor or regulatory element, e.g., an endogenous mouse CD276 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse CD276 nucleotide sequence (e.g., a portion of exon 3 and a portion of exon 4 of NM_133983.4 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse CD276 nucleotide sequence (e.g., exons 1-2, a portion of exon 3, a portion of exon 4, and exons 5-8 of NM_133983.4 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human CD276 nucleotide sequence (e.g., exons 1-2, a portion of exon 3, a portion of exon 6, and exons 7-10 of NM_001024736.2 (SEQ ID NO: 3)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human CD276 nucleotide sequence (e.g., a portion of exon 3, exons 4-5, and a portion of exon 6 of NM_001024736.2 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse CD276 amino acid sequence (e.g., an amino acid sequence encoded by a portion of exon 3 and a portion of exon 4 of NM_133983.4 (SEQ ID NO: 1); or amino acids 34-199 of NP_598744.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse CD276 amino acid sequence (e.g., an amino acid sequence encoded by exons 1-2, a portion of exon 3, a portion of exon 4, and exons 5-8 of NM_133983.4 (SEQ ID NO: 1); or amino acids 1-33 and 200-316 of NP_598744.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human CD276 amino acid sequence (e.g., an amino acid sequence encoded by exons 1-2, a portion of exon 3, a portion of exon 6, and exons 7-10 of NM_001024736.2 (SEQ ID NO: 3); or amino acids 1-33 and 418-534 of NP_001019907.1 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human CD276 amino acid sequence (e.g., an amino acid sequence encoded by a portion of exon 3, exons 4-5, and a portion of exon 6 of NM_001024736.2 (SEQ ID NO: 3); or amino acids 34-417 of NP_001019907.1 (SEQ ID NO: 4)).

The present disclosure also provides a humanized CD276 mouse amino acid sequence, wherein the amino acid sequence is selected from the group consisting of:

a) an amino acid sequence shown in SEQ ID NO: 2, 4, or 13;

b) an amino acid sequence having a homology of at least 90% with or at least 90% identical to the amino acid sequence shown in SEQ ID NO: 2, 4, or 13;

c) an amino acid sequence encoded by a nucleic acid sequence, wherein the nucleic acid sequence is able to hybridize to a nucleotide sequence encoding the amino acid shown in SEQ ID NO: 2, 4, or 13 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 2, 4, or 13;

e) an amino acid sequence that is different from the amino acid sequence shown in SEQ ID NO: 2, 4, or 13 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 2, 4, or 13.

The present disclosure also relates to a CD276 nucleic acid (e.g., DNA or RNA) sequence, wherein the nucleic acid sequence can be selected from the group consisting of:

a) a nucleic acid sequence as shown in SEQ ID NO: 1, 3, 5, 6, 7, 8, 9, 10, 11, or 12; a nucleic acid sequence encoding a homologous CD276 amino acid sequence of a humanized mouse;

b) a nucleic acid sequence that is shown in SEQ ID NO: 1, 3, 5, 6, 7, 8, 9, 10, 11, or 12;

c) a nucleic acid sequence that is able to hybridize to the nucleotide sequence as shown in SEQ ID NO: 1, 3, 5, 6, 7, 8, 9, 10, 11, or 12 under a low stringency condition or a strict stringency condition;

d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 1, 3, 5, 6, 7, 8, 9, 10, 11, or 12;

e) a nucleic acid sequence that encodes an amino acid sequence, wherein the amino acid sequence has a homology of at least 90% with or at least 90% identical to the amino acid sequence shown in SEQ ID NO: 2, 4, or 13;

f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 2, 4, or 13;

g) a nucleic acid sequence that encodes an amino acid sequence, wherein the amino acid sequence is different from the amino acid sequence shown in SEQ ID NO: 2, 4, or 13 by no more than 10, 9, 8, 7, 6, 5, 4, 3, 2 or no more than 1 amino acid; and/or

h) a nucleic acid sequence that encodes an amino acid sequence, wherein the amino acid sequence comprises a substitution, a deletion and/or insertion of one or more amino acids to the amino acid sequence shown in SEQ ID NO: 2, 4, or 13.

The present disclosure also relates to a CD276 protein sequence, wherein the amino acid sequence of the CD276 protein can be selected from the group consisting of:

a) all or part of the amino acid sequence shown in SEQ ID NO: 13; or amino acids 34-417, 32-456, 30-459, or 26-459 of SEQ ID NO: 4;

b) an amino acid sequence having a homology of at least 90%, 91%, 92%, 93%, 94%, 95%, 96%, 97%, 98%, or 99% or at least 90%, 91%, 92%, 93%, 94%, 95%, 96%, 97%, 98%, or 99% identical to the amino acid sequence shown in SEQ ID NO: 13; or amino acids 34-417, 32-456, 30-459, or 26-459 of SEQ ID NO: 4;

c) an amino acid sequence that is different from the amino acid sequence shown in SEQ ID NO: 13; or amino acids 34-417, 32-456, 30-459, or 26-459 of SEQ ID NO: 4, by no more than 10, 9, 8, 7, 6, 5, 4, 3, 2 or no more than 1 amino acid; and

d) an amino acid sequence that comprises a substitution, a deletion and/or insertion of one or more amino acids to the amino acid sequence shown in SEQ ID NO: 13; or amino acids 34-417, 32-456, 30-459, or 26-459 of SEQ ID NO: 4.

The present disclosure also relates to a humanized CD276 gene sequence, wherein the transcribed mRNA sequence of the humanized CD276 gene can be selected from the group consisting of:

a) all or part of the nucleotide sequence shown in SEQ ID NO: 12 or nucleic acids 226-1500,220-1509, 208-1509, or 232-1382 of SEQ ID NO: 3;

b) a nucleotide sequence that at least 70%, 80%, 90%, 91%, 92%, 93%, 94%, 95%, 96%, 97%, 98% or at least 99% identical to the nucleotide sequence shown in SEQ ID NO: 12 or nucleic acids 232-1382 of SEQ ID NO: 3;

c) a nucleotide sequence that is different from the nucleotide sequence shown in SEQ ID NO: 12 or nucleic acids 232-1382 of SEQ ID NO: 3 by no more than 10, 9, 8, 7, 6, 5, 4, 3, 2 or no more than 1 nucleotide; and

d) a nucleotide sequence that comprises a substitution, a deletion and/or insertion of one or more amino acids to the nucleotide sequence shown at SEQ ID NO: 12 or nucleic acids 232-1382 of SEQ ID NO: 3.

The present disclosure also relates to a humanized CD276 gene sequence, wherein the humanized CD276 gene can be selected from the group consisting of:

a) all or part of the nucleotide sequence shown in SEQ ID NO: 5;

b) a nucleotide sequence that at least 70%, 80%, 90%, 91%, 92%, 93%, 94%, 95%, 96%, 97%, 98% or at least 99% identical to the nucleotide sequence shown in SEQ ID NO: 5;

c) a nucleotide sequence that is different from the nucleotide sequence shown in SEQ ID NO: 5 by no more than 10, 9, 8, 7, 6, 5, 4, 3, 2 or no more than 1 nucleotide; and

d) a nucleotide sequence that comprises a substitution, a deletion and/or insertion of one or more amino acids to the nucleotide sequence shown at SEQ ID NO: 5.

The present disclosure further relates to a CD276 genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 5.

The disclosure also provides an amino acid sequence that has a homology of at least 90% with, or at least 90% identical to the sequence shown in SEQ ID NO: 2, 4, or 13, and has protein activity. In some embodiments, the homology with the sequence shown in SEQ ID NO: 2, 4, or 13 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 2, 4, or 13 is at least about 90%, 91%, 92%, 93%, 94%, 95%, 96%, 97%, 98%, or at least 99%.

In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 1, 3, or 12, and encodes a polypeptide that has protein activity. In some embodiments, the homology with the sequence shown in SEQ ID NO: 1, 3, or 12 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 1, 3, or 12 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For illustration purposes, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) CD276 from an endogenous non-human CD276 locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous CD276 locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wildtype nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized CD276 gene or a humanized CD276 nucleic acid. In some embodiments, at least one or more portions of the gene or the nucleic acid is from the human CD276 gene, at least one or more portions of the gene or the nucleic acid is from a non-human CD276 gene. In some embodiments, the gene or the nucleic acid comprises a sequence that encodes a CD276 protein. The encoded CD276 protein is functional or has at least one activity of the human CD276 protein or the non-human CD276 protein, e.g., binding with human or non-human CD276 receptor (e.g., TLT-2); regulating cancer progression (e.g., migration, invasion, and angiogenesis); regulating T cell activation and cytokine (e.g., IFN-7) production; increasing proliferation of CD4+ and/or CD8+ T cells; enhancing cytotoxic T lymphocyte (CTL) activity; promoting osteoblastic differentiation and bone mineralization; upregulating or downregulating the immune response.

In some embodiments, the chimeric protein or the chimeric polypeptide is a humanized CD276 protein or a humanized CD276 polypeptide. In some embodiments, at least one or more portions of the amino acid sequence of the protein or the polypeptide is from a human CD276 protein, and at least one or more portions of the amino acid sequence of the protein or the polypeptide is from a non-human CD276 protein. The humanized CD276 protein or the humanized CD276 polypeptide is functional or has at least one activity of the human CD276 protein or the non-human CD276 protein.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rodent. In some embodiments, the rodent is selected from BALB/c, A, A/He, A/J, A/WySN, AKR, AKR/A, AKR/J, AKR/N, TA1, TA2, RF, SWR, C3H, C57BR, SJL, C57L, DBA/2. KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr and C57BL/Ola C57BL, C58, CBA/Br, CBA/Ca, CBA/J, CBA/st, CBA/H strains of mice and NOD, NOD/SCID, NOD-Prkdc^{scid} IL-2rg^{null} Background mice.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized CD276 animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD-Prkdcscid IL-2rγ^{null} NOD mice, NOD-Rag 1−/−-IL2rg−/− (NRG) mice, Rag 2−/−-IL2rg−/−(RG) mice, SCID mice, NOD/SCID mice, IL2Rγ knockout mice, NOD/SCID/γc^{null} mice (Ito, M. et al., NOD/SCID/γc null mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human CD276 locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD-Prkdcscid IL-2rγ^{null} NOD mice, NOD-Rag 1−/−-IL2rg−/−(NRG) mice, Rag 2−/−-IL2rg−/−(RG) mice, NOD mice, SCID mice, NOD/SCID mice, IL-2RT knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety.

In some embodiments, the mouse can include a replacement of all or part of mature CD276 coding sequence with human mature CD276 coding sequence.

Genetically modified non-human animals that comprise a modification of an endogenous non-human CD276 locus. In some embodiments, the modification can comprise a human nucleic acid sequence encoding at least a portion of a mature CD276 protein (e.g., at least 10%, 20%, 30%, 40%, 50%, 60%, 70%, 80%, 90%, 95%, 96%, 97%, 98%, or 99% identical to the mature CD276 protein sequence). Although genetically modified cells are also provided that can comprise the modifications described herein (e.g., ES cells, somatic cells), in many embodiments, the genetically modified non-human animals comprise the modification of the endogenous CD276 locus in the germline of the animal.

Genetically modified animals can express a human CD276 and/or a chimeric (e.g., humanized) CD276 from endogenous mouse loci, wherein the endogenous mouse CD276 gene has been replaced with a human CD276 gene and/or a nucleotide sequence that encodes a region of human CD276 sequence or an amino acid sequence that is at least 10%, 20%, 30%, 40%, 50%, 60%, 70&, 80%, 90%, 95%, 96%, 97%, 98%, or 99% identical to the human CD276 sequence. In various embodiments, an endogenous non-human CD276 locus is modified in whole or in part to comprise human nucleic acid sequence encoding at least one protein-coding sequence of a mature CD276 protein.

In some embodiments, the genetically modified mice express the human CD276 and/or chimeric CD276 (e.g., humanized CD276) from endogenous loci that are under control of mouse promoters and/or mouse regulatory elements. The replacement(s) at the endogenous mouse loci provide non-human animals that express human CD276 or chimeric CD276 (e.g., humanized CD276) in appropriate cell types and in a manner that does not result in the potential pathologies observed in some other transgenic mice known in the art. The human CD276 or the chimeric CD276 (e.g., humanized CD276) expressed in animal can maintain one or more functions of the wild-type mouse or human CD276 in the animal. For example, human or non-human CD276 receptors can bind to the expressed CD276, upregulate or downregulate immune response, e.g., upregulate or downregulate immune response by at least 10%, 20%, 30%, 40%, or 50%. Furthermore, in some embodiments, the animal does not express endogenous CD276. As used herein, the term “endogenous CD276” refers to CD276 protein that is expressed from an endogenous CD276 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human CD276 (NP_001019907.1) (SEQ ID NO: 4). In some embodiments, the genome comprises a sequence encoding an amino acid sequence that is at least 70%, 75%, 80%, 85%, 90%, 95%, 99%, or 100% identical to SEQ ID NO: 4.

The genome of the genetically modified animal can comprise a replacement at an endogenous CD276 gene locus of a sequence encoding a region of endogenous CD276 with a sequence encoding a corresponding region of human CD276. In some embodiments, the sequence that is replaced is any sequence within the endogenous CD276 gene locus, e.g., exon 1, exon 2, exon 3, exon 4, exon 5, exon 6, exon 7, exon 8, 5′-UTR, 3′-UTR, the first intron, the second intron, and the third intron, the fourth intron, the fifth intron, the sixth intron, the seventh intron, etc. In some embodiments, the sequence that is replaced is within the regulatory region of the endogenous CD276 gene. In some embodiments, the sequence that is replaced starts within exon 3 and ends within exon 4 of an endogenous mouse CD276 gene locus.

The genetically modified animal can have one or more cells expressing a human or chimeric CD276 (e.g., humanized CD276) having an extracellular region and a cytoplasmic region, wherein the extracellular region comprises a sequence that is at least 50%, 60%, 70%, 80%, 90%, 95%, 99% identical to the extracellular region of human CD276. In some embodiments, the extracellular region of the humanized CD276 has a sequence that has at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, or 430 amino acids (e.g., contiguously or non-contiguously) that are identical to human CD276. Because human CD276 and non-human CD276 (e.g., mouse CD276) sequences, in many cases, are different, antibodies that bind to human CD276 will not necessarily have the same binding affinity with non-human CD276 or have the same effects to non-human CD276. Therefore, the genetically modified animal having a human or a humanized extracellular region can be used to better evaluate the effects of anti-human CD276 antibodies in an animal model. In some embodiments, the genome of the genetically modified animal comprises a sequence encoding an amino acid sequence that corresponds to part or the entire sequence of exons 3-6 of human CD276, part or the entire sequence of extracellular region of human CD276 (with or without signal peptide), or part or the entire sequence of amino acids 34-417 of SEQ ID NO: 4.

In some embodiments, the non-human animal can have, at an endogenous CD276 gene locus, a nucleotide sequence encoding a chimeric human/non-human CD276 polypeptide, wherein a human portion of the chimeric human/non-human CD276 polypeptide comprises a portion of human CD276 extracellular domain, and wherein the animal expresses a functional CD276 on a surface of a cell of the animal.

In some embodiments, the human portion of the chimeric human/non-human CD276 polypeptide comprises an amino acid sequence encoded by a portion of exon 3, exons 4-5, and a portion of exon 6 of human CD276. In some embodiments, the human portion of the chimeric human/non-human CD276 polypeptide comprises an amino acid sequence encoded by a nucleotide sequence at the 3′ end of human CD276 exon 3 encoding at least 1, 2, 3, 4, 5, 6, 7, 8, 9, 10, 11, 12, 13, 14, 15, 16, 17, 18, 19, 20, 30, 40, 50, 60, 70, 80, 90, or 100 amino acids. In some embodiments, the human portion of the chimeric human/non-human CD276 polypeptide does not comprise an amino acid sequence encoded by a nucleotide sequence at the 5′ end of human CD276 exon 3 encoding at least 1, 2, 3, 4, 5, 6, 7, 8, 9, 10, 11, 12, 13, 14, 15, 16, 17, 18, 19, or 20 amino acids. In some embodiments, the human portion of the chimeric human/non-human CD276 polypeptide comprises an amino acid sequence encoded by a nucleotide sequence at the 5′ end of human CD276 exon 6 encoding at least 1, 2, 3, 4, 5, 6, 7, 8, 9, 10, 11, 12, 13, 14, 15, 16, 17, 18, 19, 20, 30, 40, 50, or 60 amino acids. In some embodiments, the human portion of the chimeric human/non-human CD276 polypeptide does not comprise an amino acid sequence encoded by a nucleotide sequence at the 3′ end of human CD276 exon 6 encoding at least 1, 2, 3, 4, 5, 6, 7, 8, 9, 10, 11, 12, 13, 14, 15, 16, 17, 18, 19, or 20 amino acids. In some embodiments, the human portion of the chimeric human/non-human CD276 polypeptide comprises a sequence that is at least 80%, 85%, 90%, 95%, or 99% identical to amino acids 34-417 or 32-456 of SEQ ID NO: 4.

In some embodiments, the human portion of the chimeric human/non-human CD276 polypeptide comprises a portion of exon 3, exons 4-6, and a portion of exon 7 of human CD276. In some embodiments, the human portion of the chimeric human/non-human CD276 polypeptide can comprise a sequence that is at least 80%, 85%, 90%, 95%, or 99% identical to amino acids 30-459 of SEQ ID NO: 4.

In some embodiments, the human portion of the chimeric human/non-human CD276 polypeptide comprises a portion of exon 2, exons 3-6, and a portion of exon 7 of human CD276. In some embodiments, the human portion of the chimeric human/non-human CD276 polypeptide can comprise a sequence that is at least 80%, 85%, 90%, 95%, or 99% identical to amino acids 26-459 of SEQ ID NO: 4.

In some embodiments, the non-human portion of the chimeric human/non-human CD276 polypeptide comprises transmembrane and/or cytoplasmic regions of an endogenous non-human CD276 polypeptide. There may be several advantages that are associated with the transmembrane and/or cytoplasmic regions of an endogenous non-human CD276 polypeptide. For example, once a CD276 receptor (e.g., TLT-2) or an anti-CD276 antibody binds to CD276, they can properly transmit extracellular signals into the cells and initiate the downstream pathway. A human or humanized transmembrane and/or cytoplasmic regions may not function properly in non-human animal cells. In some embodiments, a few (e.g., 1, 2, 3, 4, 5, 6, 7, 8, 9, or 10) extracellular amino acids that are close to the transmembrane region of CD276 are also derived from endogenous sequence. These amino acids can also be important for transmembrane signal transmission. In some embodiments, a few (e.g., 1, 2, 3, 4, 5, 6, 7, 8, 9, or 10) amino acids at the N-terminus of the extracellular region are also derived from endogenous sequence.

Furthermore, the genetically modified animal can be heterozygous with respect to the replacement at the endogenous CD276 locus, or homozygous with respect to the replacement at the endogenous CD276 locus.

In some embodiments, the humanized CD276 locus lacks a human CD276 5′-UTR. In some embodiments, the humanized CD276 locus comprises a rodent (e.g., mouse) 5′-UTR. In some embodiments, the humanization comprises a human 3′-UTR. In appropriate cases, it may be reasonable to presume that the mouse and human CD276 genes appear to be similarly regulated based on the similarity of their 5′-flanking sequence. As shown in the present disclosure, humanized CD276 mice that comprise a replacement at an endogenous mouse CD276 locus, which retain mouse regulatory elements but comprise a humanization of CD276 encoding sequence, do not exhibit pathologies. Both genetically modified mice that are heterozygous or homozygous for humanized CD276 are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized CD276 gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized CD276 in the genome of the animal.

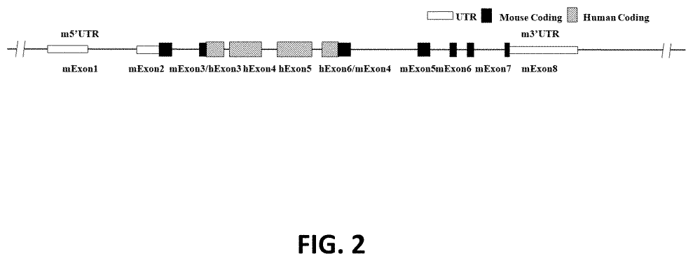

In some embodiments, the non-human mammal comprises the genetic construct as described herein (e.g., gene construct as shown in FIG. 2). In some embodiments, a non-human mammal expressing human or humanized CD276 is provided. In some embodiments, the tissue-specific expression of human or humanized CD276 protein is provided.

In some embodiments, the expression of human or humanized CD276 in a genetically modified animal is controllable, as by the addition of a specific inducer or repressor substance.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human CD276 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized CD276 protein.

### Vectors

The present disclosure relates to a targeting vector, comprising: a) a DNA fragment homologous to the 5′ end of a region to be altered (5′ arm), which is selected from the CD276 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the CD276 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000075.6; c) the DNA fragment homologous to the 3′ end of the region to be altered (3′ arm) is selected from the nucleotide sequences that have at least 90% homology to the NCBI accession number NC_000075.6.

In some embodiments, a) the DNA fragment homologous to the 5′ end of a region to be altered (5′ arm) is selected from the nucleotides from the position 58537588 to the position 58541650 of the NCBI accession number NC_000075.6; c) the DNA fragment homologous to the 3′ end of the region to be altered (3′ arm) is selected from the nucleotides from the position 58529690 to the position 58534897 of the NCBI accession number NC_000075.6.

In some embodiments, the length of the selected genomic nucleotide sequence in the targeting vector can be about 1 kB, about 1.5 kb, about 2 kb, about 2.5 kb, about 3 kb, about 3.5 kb, or about 4 kb.

In some embodiments, the region to be altered is exon 1, exon 2, exon 3, exon 4, exon 5, exon 6, exon 7, and/or exon 8 of an endogenous CD276 gene (e.g., a sequence starting within exon 3 and ending within exon 4 of mouse CD276 gene).

The targeting vector can further include a selected gene marker.

In some embodiments, the sequence of the 5′ arm is shown in SEQ ID NO: 6; and the sequence of the 3′ arm is shown in SEQ ID NO: 7. In some embodiments, the sequence of the selected genomic nucleotide sequence is shown in SEQ ID NO: 5.

In some embodiments, the sequence is derived from human (e.g., 73702275-73704354 of NC_000015.10). For example, the target region in the targeting vector is a part or entirety of the nucleotide sequence of a human CD276, preferably a sequence starting within exon 3 and ending within exon 6 of the human CD276. In some embodiments, the nucleotide sequence of the humanized CD276 encodes the entire or the part of human CD276 protein with the NCBI accession number NP_001019907.1 (SEQ ID NO: 4).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous CD276 gene locus, a sequence encoding a region of an endogenous CD276 with a sequence encoding a corresponding region of human or chimeric CD276. In some embodiments, the replacement occurs in a germ cell, a somatic cell, a blastocyst, or a fibroblast, etc. The nucleus of a somatic cell or the fibroblast can be inserted into an enucleated oocyte.

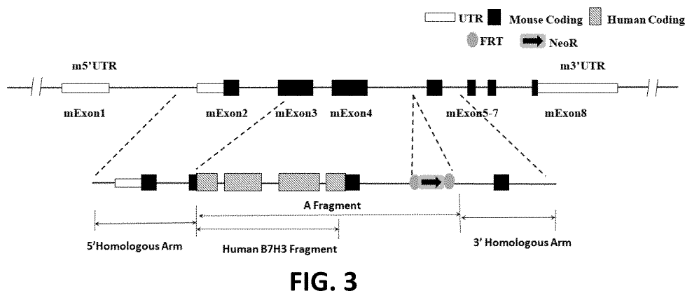

FIG. 3 shows a humanization strategy for a mouse CD276 locus. In FIG. 3, the targeting strategy involves a vector comprising the 5′ end homologous arm, human CD276 gene fragment, 3′ homologous arm. The process can involve replacing endogenous CD276 sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous CD276 sequence with human CD276 sequence.

Thus, in some embodiments, the methods for making a genetically modified, humanized animal, can include the step of replacing at an endogenous CD276 locus (or site), a sequence encoding a region of endogenous CD276 with a sequence encoding a corresponding region of human CD276. The sequence can include a region (e.g., a part or the entire region) of exon 1, exon 2, exon 3, exon 4, exon 5, exon 6, exon 7, exon 8, exon 9, and/or exon 10 of a human CD276 gene. In some embodiments, the sequence encoding a corresponding region of human CD276 includes a portion of exon 3, exons 4-5, and a portion of exon 6 of a human CD276 gene (e.g., a sequence encoding amino acids 34-417 of SEQ ID NO: 4). In some embodiments, the region is located within the extracellular region of CD276. In some embodiments, the sequence encoding a region of endogenous CD276 includes a portion of exon 3 and a portion of exon 4 of mouse CD276.

In some embodiments, the methods of modifying a CD276 locus of a mouse to express a chimeric human/mouse CD276 peptide can include the steps of replacing at the endogenous mouse CD276 locus a nucleotide sequence encoding a mouse CD276 with a nucleotide sequence encoding a human CD276, thereby generating a sequence encoding a chimeric human/mouse CD276.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a CD276 gene humanized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female non-human mammal, allowing the cell to develop in the uterus of the female non-human mammal;

(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized CD276 protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized CD276, which are useful for testing agents that can decrease or block the interaction between CD276 and CD276 receptors (e.g., TLT-2) or the interaction between CD276 and anti-human CD276 antibodies, testing whether an agent can increase or decrease the immune response, and/or determining whether an agent is an CD276 agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor or a blood cell tumor (e.g., a lymphocyte tumor, e.g., a B or T cell tumor).

In some embodiments, the genetically modified animals can be used for determining effectiveness of an anti-CD276 antibody for the treatment of cancer. The methods involve administering the anti-CD276 antibody (e.g., anti-human CD276 antibody) to the animal as described herein, wherein the animal has a tumor; and determining the inhibitory effects of the anti-CD276 antibody to the tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT.

In some embodiments, the tumor comprises one or more cancer cells (e.g., human or mouse cancer cells) that are injected into the animal. In some embodiments, the anti-CD276 antibody prevents CD276 receptors from binding to CD276. In some embodiments, the anti-CD276 antibody does not prevent CD276 receptors from binding to CD276. Exemplary anti-CD276 antibodies or antibody-drug conjugates thereof include, but not limited to, Enoblituzumab (MGA271), Omburtamab, MGD009, MGC-018, and DS-7300a.

In some embodiments, the genetically modified animals can be used for determining whether an anti-CD276 antibody is a CD276 agonist or antagonist. In some embodiments, the methods as described herein are also designed to determine the effects of the agent (e.g., anti-CD276 antibodies) on CD276, e.g., whether the agent can stimulate immune cells or inhibit immune cells (e.g., macrophages, B cells, or DC), whether the agent can increase or decrease the production of cytokines, whether the agent can activate or deactivate immune cells (e.g., macrophages, B cells, or DC), whether the agent can upregulate the immune response or downregulate immune response, and/or whether the agent can induce complement mediated cytotoxicity (CMC) or antibody dependent cellular cytoxicity (ADCC). In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-CD276 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer types as described herein include, but not limited to, lymphoma, non-small cell lung cancer (NSCLC), leukemia, ovarian cancer, nasopharyngeal cancer, breast cancer, endometrial cancer, colon cancer, rectal cancer, stomach cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, and sarcoma. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T cell lymphoma, and Waldenstrom macroglobulinemia. In some embodiments, the sarcoma is selected from osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma.

In some embodiments, the antibody is designed for treating various immune disorder or immune-related diseases (e.g., psoriasis, allergic rhinitis, sinusitis, asthma, rheumatoid arthritis, atopic dermatitis, chronic obstructive pulmonary disease (COPD), chronic bronchitis, emphysema, eczema, osteoarthritis, rheumatoid arthritis, systemic lupus erythematosus, polymyalgia rheumatica, autoimmune hemolytic anemia, systemic vasculitis, pernicious anemia, inflammatory bowel disease, ulcerative colitis, Crohn's disease, or multiple sclerosis). Thus, the methods as described herein can be used to determine the effectiveness of an anti-CD276 antibody in inhibiting immune response.

In some embodiments, the immune disorder or immune-related diseases described here include allergy, asthma, myocarditis, nephritis, hepatitis, systemic lupus erythematosus, rheumatoid arthritis, scleroderma, hyperthyroidism, primary thrombocytopenic purpura, autoimmune hemolytic anemia, ulcerative colitis, self-immune liver disease, diabetes, pain, or neurological disorders.

In some embodiments, the antibody is designed for reducing inflammation (e.g., inflammatory bowel disease, chronic inflammation, asthmatic inflammation, periodontitis, or wound healing). Thus, the methods as described herein can be used to determine the effectiveness of an antibody for reducing inflammation. In some embodiments, the inflammation described herein includes degenerative inflammation, exudative inflammation, serous inflammation, fibrinitis, suppurative inflammation, hemorrhagic inflammation, necrotitis, catarrhal inflammation, proliferative inflammation, specific inflammation, tuberculosis, syphilis, leprosy, or lymphogranuloma.

In some embodiments, the antibody is designed for treating disorders of bone mineralization, e.g., rickets, renal diseases (renal osteodystrophy, Fanconi syndrome), tumor-induced osteomalacia, hypophosphatasia, McCune-Albright syndrome, or osteogenesis imperfecta with mineralization defect (syndrome resembling osteogenesis imperfecta (SROI). In some embodiments, the disorder of bone mineralization is osteoporosis.

The present disclosure also provides methods of determining toxicity of an antibody (e.g., anti-CD276 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the CD276 gene function, human CD276 antibodies, drugs for human CD276 targeting sites, the drugs or efficacies for human CD276 targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the CD276 gene humanized non-human animal prepared by the methods described herein, the CD276 gene humanized non-human animal described herein, the double- or multi-humanized non-human animal generated by the methods described herein (or progeny thereof), a non-human animal expressing the human or humanized CD276 protein, or the tumor-bearing or inflammatory animal models described herein. In some embodiments, the TCR-T, CAR-T, and/or other immunotherapies can treat the CD276-associated diseases described herein. In some embodiments, the TCR-T, CAR-T, and/or other immunotherapies provides an evaluation method for treating the CD276-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric CD276 gene and a sequence encoding an additional human or chimeric protein.

In some embodiments, the additional human or chimeric protein can be programmed cell death protein 1 (PD-1), IL4, Colony Stimulating Factor 1 (CSF1), IL34, C—C Motif Chemokine Receptor 2 (CCR2), CD40, C—X—C Motif Chemokine Receptor 4 (CXCR4), Vascular Endothelial Growth Factor (VEGF), cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD27, CD28, CD47, CD137, CD154, T-Cell Immuno receptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α (SIRPα) or TNF Receptor Superfamily Member 4 (TNFRSF4 or OX40).

The methods of generating genetically modified animal model with two or more human or chimeric genes (e.g., humanized genes) can include the following steps:

(a) using the methods of introducing human CD276 gene or chimeric CD276 gene as described herein to obtain a genetically modified non-human animal;

(b) breeding the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric PD-1, IL4, CSF1, IL34, CCR2, CD40, CXCR4, VEGF, CTLA-4, LAG-3, BTLA, PD-L1, CD27, CD28, CD47, CD137, CD154, TIGIT, TIM-3, GITR, SIRPα, or OX40. Some of these genetically modified non-human animal are described, e.g., in PCT/CN2017/090320, PCT/CN2017/099577, PCT/CN2017/099575, PCT/CN2017/099576, PCT/CN2017/099574, PCT/CN2017/106024, PCT/CN2017/110494, PCT/CN2017/110435, PCT/CN2017/120388, PCT/CN2018/081628, PCT/CN2018/081629; each of which is incorporated herein by reference in its entirety.

In some embodiments, the CD276 gene humanization is directly performed on a genetically modified animal having a human or chimeric PD-1, IL4, CSF1, IL34, CCR2, CD40, CXCR4, VEGF, CTLA-4, BTLA, PD-L1, CD27, CD28, CD47, CD137, CD154, TIGIT, TIM-3, GITR, SIRPα, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-CD276 antibody and an additional therapeutic agent (e.g., an anti-PD-1 antibody) for the treatment of cancer. The methods include administering the anti-CD276 antibody and the additional therapeutic agent (e.g., an anti-PD-1 antibody) to the animal, wherein the animal has a tumor; and determining the inhibitory effects of the combined treatment to the tumor. In some embodiments, the additional therapeutic agent is an antibody that specifically binds to PD-1, IL4, CSF1, IL34, CCR2, CD40, CXCR4, VEGF, CTLA-4, BTLA, PD-L1, CD27, CD28, CD47, CD137, CD154, TIGIT, TIM-3, GITR, SIRPα, or OX40. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the combination treatment is designed for treating melanoma, carcinomas (e.g., pancreatic carcinoma), mesothelioma, hematological malignancies (e.g., Non-Hodgkin's lymphoma, lymphoma, chronic lymphocytic leukemia), or solid tumors (e.g., advanced solid tumors). In some embodiments, the combination treatment is designed for treating liver cancer, pancreatic cancer, osteosarcoma, breast cancer, ovarian cancer, endometrial cancer, oral squamous cell carcinoma, cervical cancer, renal cancer, head and neck cancer, or brain cancer.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

MfeI, NdeI, and HindIII restriction enzymes were purchased from NEB. Catalog numbers are R3589V, R0111V, and R3104V, respectively.

Lipopolysaccharides from Escherichia coli 0111:B4 was purchased from Sigma with catalog number L2630.

Attune™ Nxt Acoustic Focusing Cytometer was purchased from Thermo Fisher Scientific, with model number Attune™ Nxt.

PrimeScript™ 1st Strand cDNA Synthesis Kit was purchased from Takara Bio Inc. with catalog number 6110A.

Heraeus™ Fresco™ 21 Microcentrifuge was purchased from Thermo Fisher Scientific with model number Fresco™ 21.

Enoblituzumab was purchased from BioLegend with catalog number 343606.

Anti-mouse PD-1 antibody InVivoMab anti-mouse PD-1 (CD279) was purchased from Bio X Cell, with catalog number BE0146.

Purified anti-mouse CD16/32 Antibody was purchased from BioLegend with catalog number 101302.

Brilliant Violet 510™ anti-mouse CD45 was purchased from BioLegend with catalog number 103138.

PerCP anti-mouse Ly-6G/Ly-6C (Gr-1) Antibody was purchased from BioLegend with catalog number 108426.

Brilliant Violet 421™ anti-mouse CD4 was purchased from BioLegend with catalog number 100438.

FITC anti-mouse F4/80 was purchased from BioLegend with catalog number 123108.

PE anti-mouse CD8a Antibody was purchased from BioLegend with catalog number 100708.

PE/Cy™ 7 Mouse anti-mouse NK1.1 was purchased from BD Biosciences with catalog number 552878 (under brand BD Pharmingen™) APC anti-mouse/rat Foxp3 Antibody was purchased from eBioscience, Inc. with catalog number 17-5773-82.

FITC anti-Mouse CD19 Antibody was purchased from BioLegend with catalog number 115506.

PerCP/Cy5.5 anti-mouse TCR R chain was purchased from BioLegend with catalog number 109228.

APC Hamster Anti-Mouse TCR R chain was purchased from BD Biosciences with catalog number 553174 (under brand BD Pharmingen™)

Brilliant Violet 605™ anti-mouse CD11c was purchased from BioLegend with catalog number 117334.

PE anti-mouse/human CD1 lb was purchased from BioLegend with catalog number 101208.

### Example 1: Mice with Humanized CD276 Gene

In this example, a non-human animal (e.g., a mouse) was modified to include a nucleotide sequence encoding human CD276 protein, and the obtained genetically-modified non-human animal can express a human or humanized CD276 protein in vivo. The mouse CD276 gene (NCBI Gene ID: 102657, Primary source: MGI: 2183926, UniProt ID: Q8VE98) is located at 58524300 to 58555033 of chromosome 9 (NC_000075.6), and includes 8 exons. The human CD276 gene (NCBI Gene ID: 80381, Primary source: HGNC: 19137, UniProt ID: Q5ZPR3) is located at 73683966 to 73714518 of chromosome 15 (NC_000015.10), and includes 10 exons. The mouse CD276 transcript sequence NM_133983.4 is set forth in SEQ ID NO: 1, and the corresponding protein sequence NP_598744.1 is set forth in SEQ ID NO: 2. The human CD276 transcript sequence NM_001024736.2 is set forth in SEQ ID NO: 3, and the corresponding protein sequence NP_001019907.1 is set forth in SEQ ID NO: 4. Mouse and human CD276 gene loci are shown in FIG. 1A and FIG. 1B, respectively.

A nucleotide sequence encoding human CD276 protein was introduced into the endogenous mouse CD276 locus, such that the mouse can express a human or humanized CD276 protein. Mouse cells can be modified by various gene-editing techniques, for example, replacement of specific mouse CD276 gene sequences with human CD276 gene sequences at the endogenous mouse CD276 locus. For example, under control of a mouse CD276 regulatory element, a sequence spanning from exon 3 (including a part of exon 3) to exon 4 (including a part of exon 4) of the mouse CD276 gene can be replaced with a corresponding sequence spanning from exon 3 (including a part of exon 3) to exon 6 (including a part of exon 6) of the human CD276 gene, to obtain a mouse chimeric CD276 locus as shown in FIG. 2, thereby humanizing mouse CD276 gene.

Mouse CD276 DNA was obtained using Bacterial Artificial Chromosome (BAC) RP23-92M21, and human CD276 gene was directly synthesized. As shown in the schematic diagram of the targeting strategy in FIG. 3, the targeting vector includes an upstream homologous arm, a downstream homologous arm, and an “A fragment” including a human CD276 gene sequence. Sequence of the upstream homologous arm (5′ homologous arm, SEQ ID NO: 6, 4064 bp) is identical to nucleotide sequence of 58537588-58541650 of NCBI accession number NC_000075.6, and sequence of the downstream homologous arm (3′ homologous arm, SEQ ID NO: 7, 5208 bp) is identical to nucleotide sequence of 58529690-58534897 of NCBI accession number NC_000075.6. The A fragment (SEQ ID NO: 5, 2080 bp) comprises a sequence spanning from exon 3 (including a part of exon 3) to exon 6 (including a part of exon 6) of a human genomic CD276 gene, which is identical to nucleotide sequence of 73702275-73704354 of NCBI accession number NC_000015.10.

The connection between the 5′ end of the human CD276 gene sequence in the A fragment and the mouse CD276 gene locus was designed as: 5′-TCACCCCCAGGAGCTGTGGAAGTCCAGGTCCCTGAAGACCCAGTGGTGGCACTG GTGGGCA-3′ (SEQ ID NO: 8), wherein the “C” of the sequence “GGTC” is the last nucleotide of the mouse sequence, and the first “C” of the sequence “CCTG” is the first nucleotide of the human sequence. The connection between the 3′ end of the human CD276 gene sequence and the mouse CD276 gene locus was designed as: 5′-ACTGGCAACGTGACCACGTCGCAGATGGCCAACGAGCAGGGCTTGTTCGATGT TCACAGCGTGCTGAGGGT-3′ (SEQ ID NO: 9), wherein the last “G” of the sequence “GCAG” is the last nucleotide of the human sequence, and the first “G” of the sequence “GGCT” is the first nucleotide of the mouse sequence. The targeting vector also included an antibiotic resistance gene for positive clone screening (neomycin phosphotransferase gene, or Neo), and two Frt recombination sites flanking the antibiotic resistance gene, that formed a Neo cassette. The Neo cassette is located between exon 4 and exon 5 of mouse CD276 gene. The connection between the 5′ end of the Neo cassette and the mouse CD276 gene locus was designed as: 5′-CTTAAATCTCTGGTTTCCAGGTCAGCACTAGTTCAAGTAGGCACCAATTGAAGCTTG ATATCGAATTCCGAAGTTCCTATTCTCTAGAAAGTATAGGAACTTCA-3′ (SEQ ID NO: 10), wherein the last “C” of the sequence “GCAC” is the last nucleotide of the mouse sequence, and the first “C” of the sequence “CAAT” is the first nucleotide of the Neo cassette. The connection between the 3′ end of the Neo cassette with the mouse CD276 sequence was designed as 5′-AAGTTCCTATTCTCTAGAAAGTATAGGAACTTCATCAGTCAGGTACATAATGGTGGA TCCCTTCAGTGCTTGGGGATCTAAAGAGGGTTCAGGCTCACCT-3′ (SEQ ID NO: 11), wherein the last “C” of the sequence “ATCC” is the last nucleotide of the Neo cassette, and the first “C” of the sequence “CTTC” is the first nucleotide of the mouse sequence. In addition, a coding gene with a negative selectable marker (a gene encoding diphtheria toxin A subunit (DTA)) was also inserted downstream of the 3′ homologous arm of the targeting vector. The mRNA sequence of the engineered mouse CD276 after humanization and its encoded protein sequence are shown in SEQ ID NO: 12 and SEQ ID NO: 13, respectively.

The targeting vector was constructed, e.g., by restriction enzyme digestion and ligation, or synthesized directly. The constructed targeting vector sequence was preliminarily verified by restriction enzyme digestion, then verified by sequencing. The correct targeting vector was electroporated and transfected into embryonic stem cells of C57BL/6 mice. The positive selectable marker gene was used to screen the cells, and the integration of exogenous genes was confirmed by PCR and Southern Blot. Specifically, positive clones identified by PCR were further confirmed by Southern Blot to screen out correct positive clone cells.

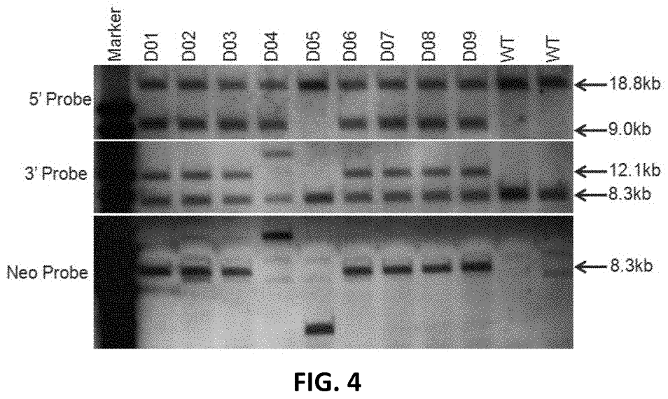

Either the primers ES-F1 and ES-R1, or primers ES-F2 and ES-R2, were used for PCR amplification, and all 9 clones were identified as positive clones with numbers D01, D02, D03, D04, D05, D06, D07, D08, and D09. The positive clones identified by PCR were then verified by Southern Blot. Specifically, genomic DNA of the positive clone cells was digested with MfeI, NdeI, or HindIII, respectively, and then hybridized with 3 corresponding probes. The lengths of the probes and target fragments are shown in the table below. As shown in FIG. 4, the results indicate that D01, D02, D03, D06, D07, D08, and D09 were positive heterozygous clones and no random insertions were detected.

TABLE 3

| | | | Recombinant |
|---|---|---|---|
| | | Wild-type | sequence |
| Restriction | | fragment | fragment |
| Enzyme | Probe | size | size |
| MfeI | 5′ Probe | 18.8 kb | 9.0 kb |
| NdeI | 3′ Probe | 8.3 kb | 12.1 kb |
| HindIII | Neo Probe | — | 8.3 kb |

The following primers were used in PCR:

| (SEQ ID NO: 14) | |
|---|---|
| ES-F1: | 5′-GCTCGACTAGAGCTTGCGGA-3′; |
| (SEQ ID NO: 15) | |
| ES-R1: | 5′-TCTGTTCATCCTCTCACAGGCACAG-3′; |
| (SEQ ID NO: 16) | |
| ES-F2: | 5′-CTGTGTACCTGAGGCTAGACCACATG-3′; |
| (SEQ ID NO: 17) | |
| ES-R2: | 5′-AGGGATGCGTTGCCCTGTGC-3′. |

The following probes were used in Southern Blot assays:

| 5′ probe: | |
|---|---|
| (SEQ ID NO: 18) | |
| 5′ Probe-F: | 5′-GGCAGCAGATGGGGCTAAGATGAAT-3′, |
| (SEQ ID NO: 19) | |
| 5′ Probe-R: | 5′-CGAAAGACTACAGAGTGCTTTGGCCT-3′; |
| 3′ probe: | |
| (SEQ ID NO: 20) | |
| 3′ Probe-F: | 5′-TGCGTTTGGGAGGGAAACAGACATT-3′, |
| (SEQ ID NO: 21) | |
| 3′ Probe-R: | 5′-AACTGTGAGGCTCTGAACATTCGGC-3′; |
| Neo probe: | |
| (SEQ ID NO: 22) | |
| Neo Probe-F: | 5′-GGATCGGCCATTGAACAAGAT-3′, |
| (SEQ ID NO: 23) | |
| Neo Probe-R: | 5′-CAGAAGAACTCGTCAAGAAGGC-3′. |

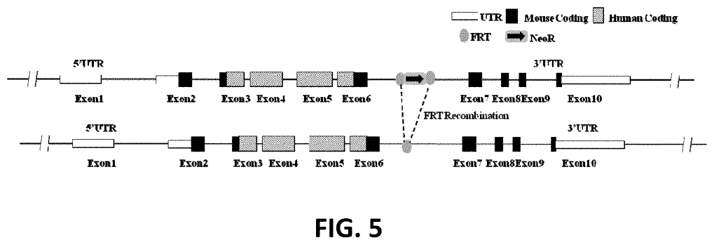

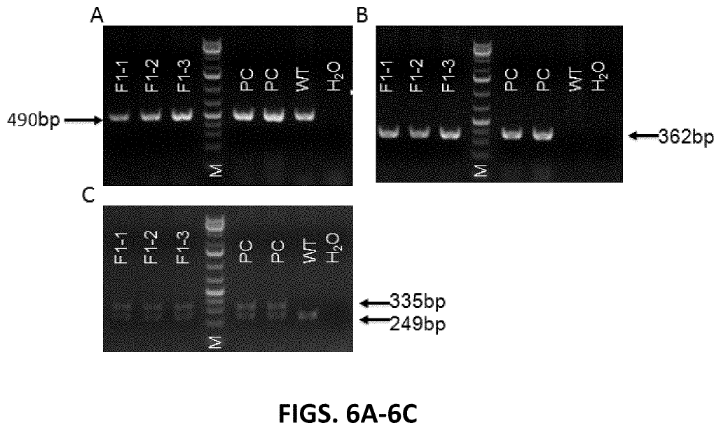

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the F1p transgenic mice to remove the positive selectable marker gene (FIG. 5), and then the humanized CD276 homozygous mice expressing humanized CD276 protein were obtained by breeding with each other. The genotype of the progeny mice can be identified by PCR using primers shown in the table below. The identification results of exemplary F1 generation mice (Neo cassette-removed) are shown in FIGS. 6A-6C, and mice labelled F1-1, F1-2, F1-3 were identified as positive heterozygous clones. Specifically, primers WT-F and WT-R were used to amplify a fragment from endogenous mouse CD276 gene (FIG. 6A); primers WT-F and Mut-R were used to amplify a fragment from modified mouse CD276 gene (FIG. 6B); and primers Frt-F and Frt-R were used to verify whether the antibiotic resistance gene was removed (FIG. 6C).

TABLE 4

| | | | Fragment |
|---|---|---|---|
| | | Tm | size |
| Primer | Primer sequence (5′-3′) | (° C.) | (bp) |
| WT-F | ACTCTGAGCCTCTGACCTGAAAGGA | 62 | WT: 490 |
| | (SEQ ID NO: 24) | | |
| WT-R | GAAGCAGAGGGTACCCCTCTCC | 61 | |
| | (SEQ ID NO: 25) | | |
| WT-F | ACTCTGAGCCTCTGACCTGAAAGGA | 62 | Mut: 362 |
| | (SEQ ID NO: 24) | | |
| Mut-R | AGGGATGCGTTGCCCTGTGC | 64 | |
| | (SEQ ID NO: 27) | | |
| Frt-F | AGACTCACACTGGAGGAGTCTCTT | 59 | WT: 249 |
| | (SEQ ID NO: 28) | | (Neo |
| | | | cassette- |
| | | | removed) |
| Frt-R | TCAGAGACAGAGAGGACCCTGGG | 61 | Mut: 335 |
| | (SEQ ID NO: 29) | | |

The above results indicated that this method can be used to construct genetically engineered CD276 mice and the genetic modification can be stably passed to the next generation without random insertions.

According to the method described above, the human portion of the humanized CD276 protein may also include amino acid residues outside of 34-417 of SEQ ID NO: 4, which are identical to the corresponding residues of endogenous CD276 protein of the non-human animal. Such residues can be derived from the extracellular region and/or signal peptide, e.g., amino acids 32-456, or amino acids 30-459 of SEQ ID NO: 4, within the extracellular region of human CD276 protein. An alignment of the amino acid resides between human and mouse CD276 is shown in FIG. 11. The mRNA sequence of the engineered mouse CD276 after humanization and its encoded protein sequence are shown in SEQ ID NO: 12 and SEQ ID NO: 13, respectively. Further, the humanized CD276 protein may also include amino acids 26-459 of SEQ ID NO: 4, spanning both the extracellular region and signal peptide of human CD276 protein.

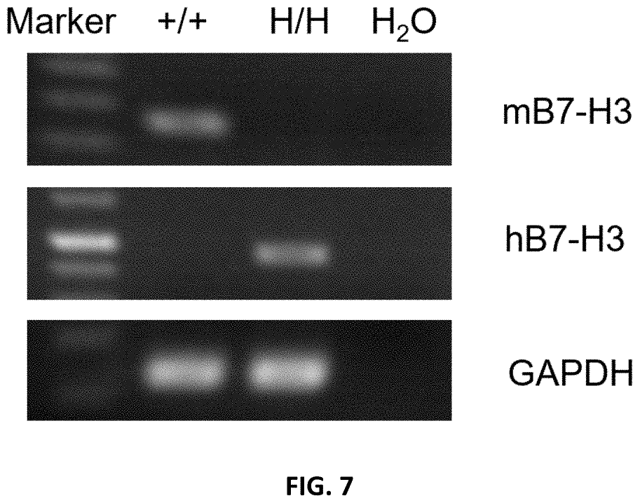

The expression of the humanized CD276 in CD276 gene humanized homozygous mice was confirmed by RT-PCR. Specifically, mouse spleen was isolated from wild-type C57BL/6 mice (+/+) or CD276 gene humanized homozygous mice (H/H), and total RNA was extracted from the spleen cells. Complementary DNA (cRNA) was then generated using a reverse transcription kit. Primers mCd276-F1 (SEQ ID NO: 30): GGACCAAGGCAGTGCCTACT, and mCd276-R1 (SEQ ID NO: 31): TGGTCACCATGTTCCCTGGACGT were used to amplify a 239 bp fragment from mouse CD276 gene; primers hCD276-F1 (SEQ ID NO: 32): CAGCTGGTGCACAGCTTTGCT and hCD276-R1 (SEQ ID NO: 33): GGCAGCTGTAGGTGCCATTTGCA were used to amplify a 442 bp fragment from human CD276 gene; primers GAPDH-F (SEQ ID NO: 34): AGGTCGGTGTGAACGGATTTG, and GAPDH-R (SEQ ID NO: 26): TGTAGACCATGTAGTTGAGGTCA were used to amplify a 123 bp fragment from GAPDH, which was used as a control. As shown in FIG. 7, mouse CD276 (mB7-H3) was detected only in wild-type C57BL/6 mice (+/+), but not in CD276 gene humanized homozygous mice (H/H). By contrast, human CD276 (hB7-H3) was detected only in CD276 gene humanized homozygous mice (H/H), but not in wild-type C57BL/6 mice (+/+). The results indicate that the humanized CD276 can be expressed in CD276 gene humanized mice.

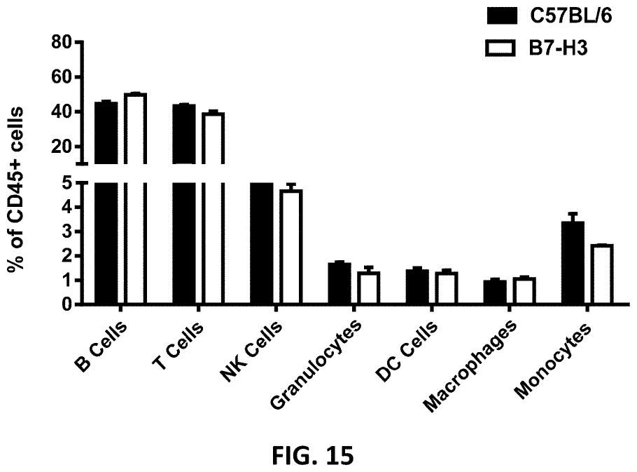

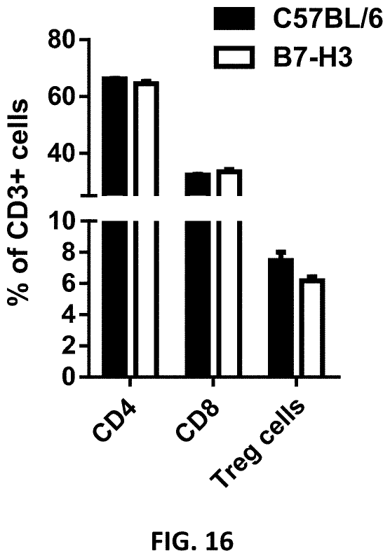

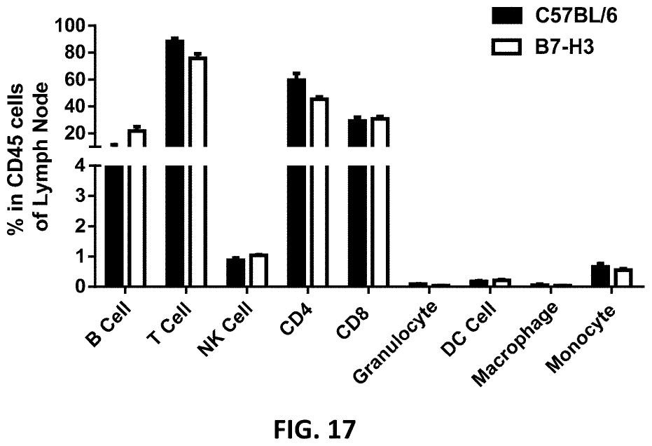

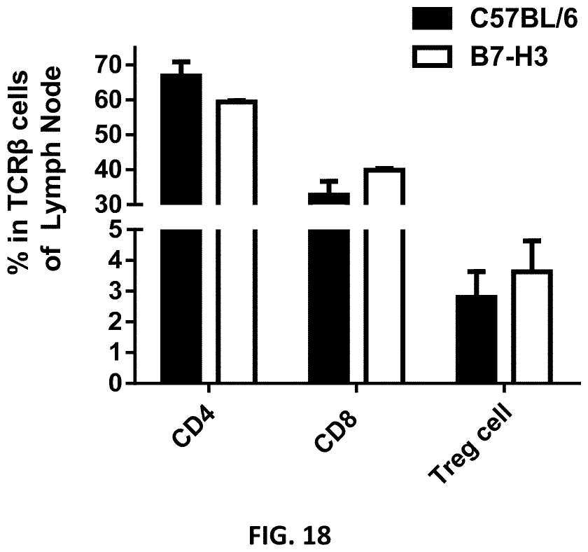

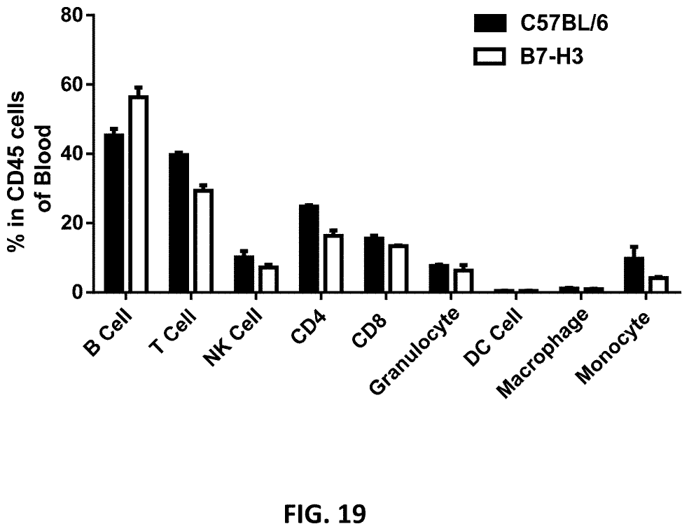

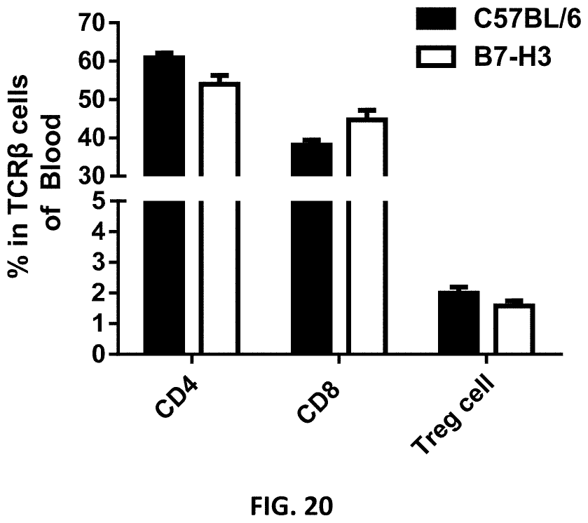

Further, immuno-phenotyping of leukocytes and T cells in the spleen of wild-type C57BL/6 mice and CD276 gene humanized homozygous mice was performed by flow cytometry. Three wild-type C57BL/6 mice (6-8 weeks old) and three CD276 gene humanized homozygous mice (6-8 weeks old) were selected. Spleen cells, lymph nodes and peripheral blood were collected from the mice after euthanasia. The cells were stained with Purified anti-mouse CD16/32 Antibody, Brilliant Violet 510™ anti-mouse CD45 (an anti-mouse CD45 antibody), PerCP anti-mouse Ly-6G/Ly-6C (Gr-1) Antibody, Brilliant Violet 421™ anti-mouse CD4 (an anti-mouse CD4 antibody), FITC anti-mouse F4/80 (an anti-mouse F4/80 antibody), PE anti-mouse CD8a Antibody, PE/Cy™ 7 Mouse anti-mouse NK1.1 (an anti-mouse NK cell surface antigen antibody), APC anti-mouse/rat Foxp3 Antibody, FITC anti-Mouse CD19 Antibody, PerCP/Cy5.5 anti-mouse TCR R chain, APC Hamster Anti-Mouse TCR R chain, Brilliant Violet 605™ anti-mouse CD11c, or PE anti-mouse/human CD11b, followed by flow cytometry detection. The detection results of leukocyte subtypes and T cell subtypes in the spleen are shown in FIG. 15 and FIG. 16, respectively. The detection results of leukocyte subtypes and T cell subtypes in the lymph nodes are shown in FIG. 17 and FIG. 18, respectively. The detection results of leukocyte subtypes and T cell subtypes in the peripheral blood are shown in FIG. 19 and FIG. 20, respectively.

The results showed that in mouse spleen, the leukocyte subtypes, e.g., B cells, T cells, NK cells, granulocytes, DC cells, macrophages, and monocytes of CD276 gene humanized homozygous mice had comparable levels as compared to those of wild-type C57BL/6 mice (FIG. 15). The results also showed that the percentages of T cell subtypes, e.g., CD4+ T cells, CD8+ T cells, and Treg cells in CD276 gene humanized homozygous mice, were comparable with those detected in wild-type C57BL/6 mice (FIG. 16).

In addition, the results showed that in lymph nodes and peripheral blood, the leukocyte subtypes, e.g., B cells, T cells, NK cells, CD4+ T cells, CD8+ T cells, granulocytes, DC cells, macrophages, and monocytes of CD276 gene humanized homozygous mice had comparable levels as compared to those of wild-type C57BL/6 mice (FIG. 17 and FIG. 19). The results also showed that the percentages of T cell subtypes, e.g., CD4+ T cells, CD8+ T cells, and Treg cells in CD276 gene humanized homozygous mice, were comparable with those detected in wild-type C57BL/6 mice (FIG. 18 and FIG. 20). The above results indicate that modification (e.g., humanization) of the mouse CD276 gene did not affect the differentiation, development, and distribution of leukocytes in the lymphatic tissues of mice.

### Example 2: Generation of Double- or Multi-Gene Humanized Mice

The humanized CD276 mouse prepared by the methods described herein can also be used to prepare a double- or multi-gene humanized mouse model. For example, in Example 1, the embryonic stem cells used for blastocyst microinjection can be selected from mice containing genetic modifications, e.g., PD-1 gene humanized mice, thereby generating CD276/PD-1 double-gene humanized mice. Alternatively, the embryonic stem cells of CD276 gene humanized mice can be selected for gene editing, to obtain a double-gene or multi-gene humanized mouse model comprising humanized CD276 and other genetic modifications. In addition, it is also possible to breed the homozygous or heterozygous CD276 transgenic mice obtained by the methods described herein with other genetically modified homozygous or heterozygous mice, and the offspring can be screened. According to Mendel's law, it is possible to generate double-gene or multi-gene modified heterozygous mice comprising humanized CD276 gene and other genetic modifications. Then the heterozygous mice can be bred with each other to obtain homozygous double-gene or multi-gene humanized mice.

For example, CD276/PD-1 double-gene humanized mice can be generated using the above method. Because mouse CD276 and PD-1 genes are located on chromosomes 9 and 1, respectively, the CD276 gene humanized mice prepared in Example 1 can be bred with PD-1 gene humanized mice, and the positive offspring can be screened, thereby generating CD276/PD-1 double-gene humanized mice.

A similar method can also be used for the generation of triple-gene humanized mice. For example, two single-gene humanized mice can be bred to generate double-gene humanized mice, and then the double-gene humanized mice can be bred with another single-gene humanized mice. Positive offspring can be screened to generate triple-gene humanized mice.

### Example 3. Pharmacological Validation of CD276 Gene Humanized Animal Model

CD276 gene humanized homozygous mice (4-5 weeks old) were subcutaneously injected with mouse colon cancer cell MC38-hCD276 (i.e., CD276 gene humanized MC38 cells), and when the tumor volume grew to about 100±50 mm³, the mice were randomly placed into a control group (G1) and a treatment group (G2) based on tumor size (5 mice per group). The treatment group mice were administered with the anti-human CD276 antibody Enoblituzumab via intraperitoneal injection (i.p.), whereas the control group mice were injected with an equal volume of an isotype control antibody (human IgG, or hIgG). The frequency of administration was twice a week (6 times of administrations in total). The tumor volume was measured twice a week and the body weight of the mice was weighed as well. Euthanasia was performed when the tumor volume of the mouse reached 3000 mm³.

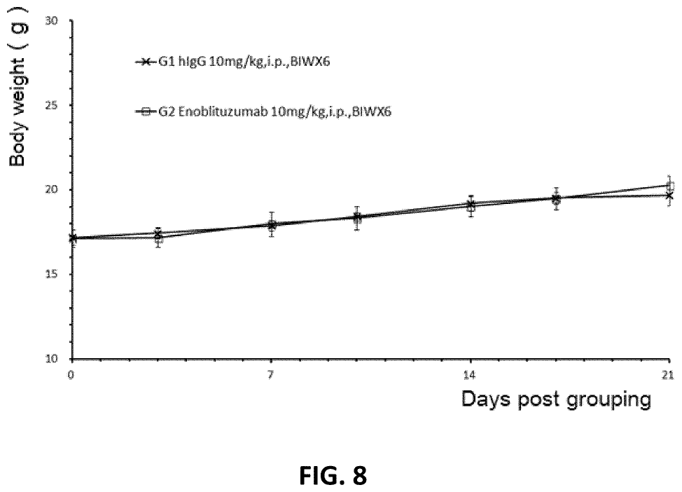

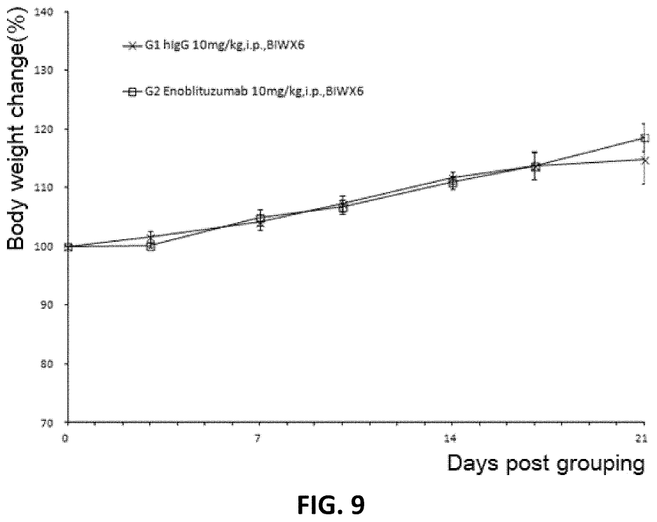

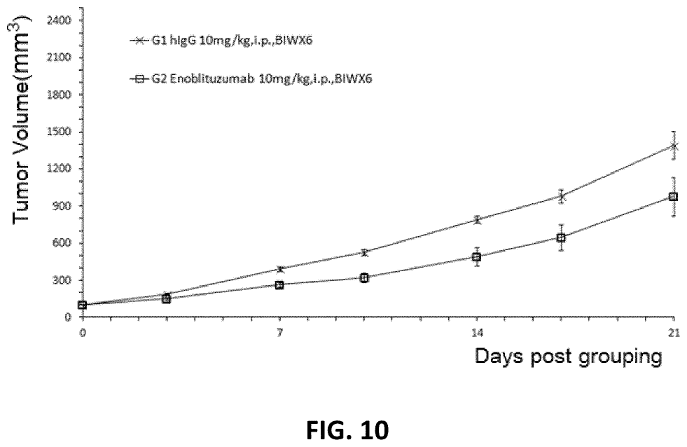

Overall, the animals in each group were healthy, and the body weights of all the treatment and control group mice increased, and were not significantly different from each other during the experimental period (FIG. 8 and FIG. 9). According to the results in FIG. 10 and the table below, the tumor volume of all control group mice continued to grow during the experimental period. At the end of the experimental period (Day 21 post grouping), the average tumor volume of the control group mice (G1) was 1393±113 mm³, whereas the average tumor volume of the treatment group mice (G2) was 980±156 mm³. The tumor volume of the treatment group mice was significantly smaller than that of the control group mice, and the tumor growth of the treatment group mice was inhibited at various degrees. The results indicate that the anti-human CD276 antibody can be used to inhibit tumor growth, and has no obvious toxic effect on animals.

TABLE 5

| | | | | Survived | Tumor- | | P value | |
|---|---|---|---|---|---|---|---|---|
| | Tumor volume (mm³) | | | mice on | free mice | | Body | Tumor |
| | Day 0 | Day 10 | Day 21 | Day 21 | on Day 21 | TGI_{TV} % | weight | volume |
| Control | 99 ± 1 | 528 ± 26 | 1393 ± 113 | 5/5 | 0/5 | N/A | N/A | N/A |
| group (G1) | | | | | | | | |
| Treatment | 99 ± 2 | 321 ± 39 | 980 ± 156 | 5/5 | 0/5 | 31.9 | 0.462 | 0.064 |
| group (G2) | | | | | | | | |

### Example 4. In Vivo Efficacy Verification of Combination Therapy

A similar experiment was performed as described in Example 3, with the dosage regimen shown in the table below. Specifically, CD276 gene humanized homozygous mice (4-5 weeks old) were subcutaneously injected with mouse colon cancer cell MC38-hCD276 (i.e., CD276 gene humanized MC38 cells), and when the tumor volume grew to about 100±50 mm³, the mice were randomly placed into a control group (G1) and three treatment groups (G2-G4) based on tumor size (6 mice per group). The treatment group mice were administered with an anti-mouse PD-1 antibody InVivoMab anti-mouse PD-1 (CD279) (G2); Enoblituzumab (G3); or the anti-mouse PD-1 antibody in combination with Enoblituzumab (G4), via intraperitoneal injection (i.p.), whereas the control group mice were injected with an equal volume of an isotype control antibody hIgG (G1). The frequency of administration was twice a week (8 times of administrations in total). The tumor volume was measured twice a week and the body weight of the mice was weighed as well. Euthanasia was performed when the tumor volume of the mouse reached 3000 mm³.

TABLE 6
Dosing regimen

| | | | | | Administration |
|---|---|---|---|---|---|
| | | Animal | | Administration | frequency and |
| Group | Antibody | number | Dose level | route | total times |
| G1 | Human IgG1 | 6 | First 6 times: 10 mg/kg | i.p. | BIW × 8 |
| | | | Last 2 times: 20 mg/kg | | |
| G2 | Anti-mouse PD-1 | 6 | First 3 times: 10 mg/kg | i.p. | BIW × 8 |
| | antibody | | Last 5 times: 3 mg/kg | | |
| G3 | Enoblituzumab | 6 | First 6 times: 10 mg/kg | i.p. | BIW × 8 |
| | | | Last 2 times: 20 mg/kg | | |
| G4 | Enoblituzumab + | 6 | Enoblituzumab: | i.p. | BIW × 8 |
| | Anti-mouse PD-1 | | First 6 times: 10 mg/kg | | |
| | antibody | | Last 2 times: 20 mg/kg | | |
| | | | Anti-mPD-1AB: | | |
| | | | First 3 times: 10 mg/kg | | |
| | | | Last 5 times: 3 mg/kg | | |
| | | | (co-administered with | | |
| | | | Enoblituzumab) | | |

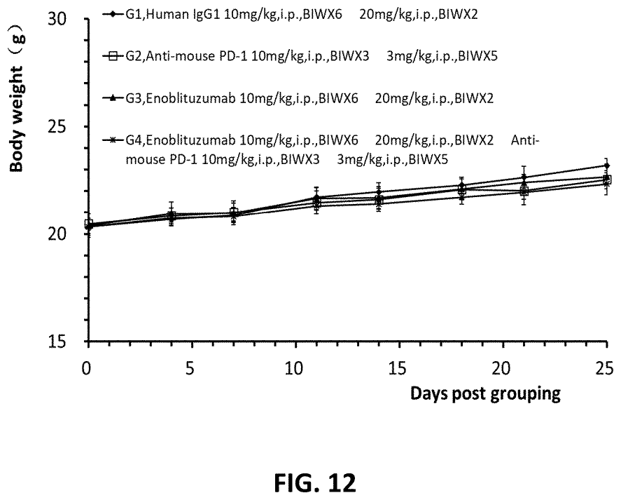

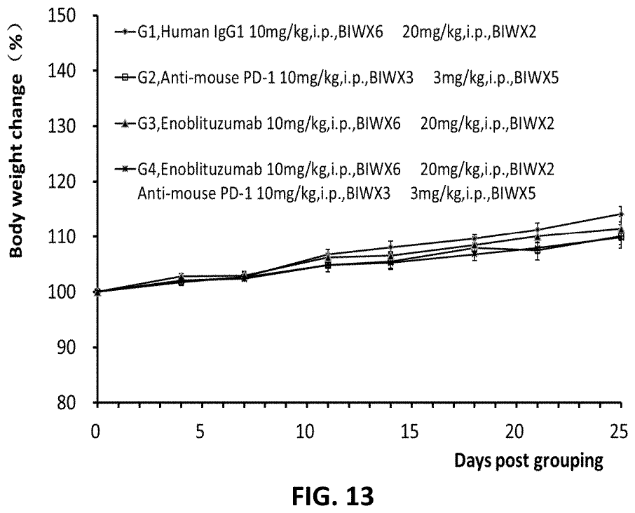

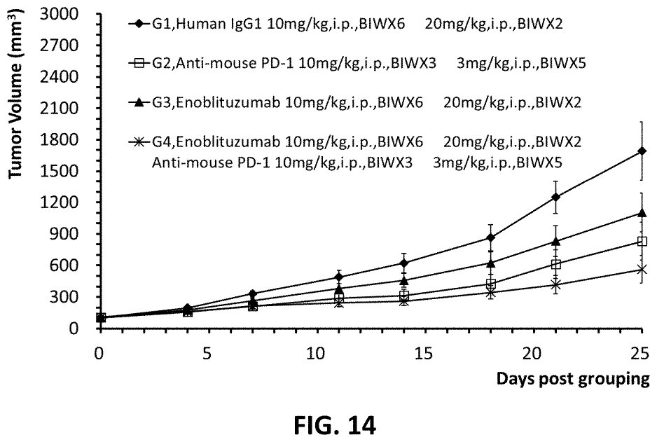

Overall, the animals in each group were healthy, and the body weights of all the treatment group mice (G2-G4) and control group mice (G1) increased, and were not significantly different from each other during the experimental period (FIG. 12 and FIG. 13). The results indicate that the treatment group mice tolerated the anti-mouse PD-1 antibody well. According to the results shown in FIG. 14 and the table below, the tumor volume of all control group mice continued to grow during the experimental period. By contrast, all treatment group mice showed different degrees of tumor growth inhibition. At the end of the experimental period (Day 25 post grouping), the average tumor volume of the control group mice (G1) was 1690±279 mm³, whereas the average tumor volume of the treatment group mice in groups G2-G4 were 829±180 mm³, 1103±185 mm³, and 563±131 mm³, respectively. Moreover, as compared to the control group (G1), the tumor volumes of the G2 and G4 group mice were significantly inhibited on Day 25 (P<0.05). With respect to the tumor treatment effect, the tumor growth inhibition rate (TGI_{TV}%) of the treatment group mice (G2, G3, and G4) were 54.3%, 37.1% and 71.2%, respectively, indicating that the tumor growth in the G4 group mice was significantly inhibited (TGI_{TV}>60).

The above results also showed that in comparison with Enoblituzumab or the anti-mouse PD-1 antibody monotherapy, the combination therapy of Enoblituzumab and the anti-mouse PD-1 antibody exhibited a synergistic effect in inhibiting tumor growth.

TABLE 7

| | | | | Survived | | P value | |
|---|---|---|---|---|---|---|---|
| | Tumor volume (mm³) | | | mice on | | Body | Tumor |
| | Day 0 | Day 14 | Day 25 | Day 25 | TGI_{TV} % | weight | volume |
| Control | 106 ± 3 | 622 ± 94 | 1690 ± 279 | 6/6 | N/A | N/A | N/A |
| group (G1) | | | | | | | |
| Treatment | 106 ± 3 | 314 ± 58 | 829 ± 180 | 6/6 | 54.3 | 0.432 | 0.027 * |
| group (G2) | | | | | | | |
| Treatment | 106 ± 4 | 458 ± 65 | 1103 ± 185 | 6/6 | 37.1 | 0.278 | 0.110 |
| group (G3) | | | | | | | |
| Treatment | 106 ± 4 | 261 ± 44 | 563 ± 131 | 6/6 | 71.2 | 0.067 | 0.004 ** |
| group (G4) | | | | | | | |

Note:
* P value < 0.05;
** P value < 0.01.

The above experiments demonstrated that the humanized CD276 mice prepared by the methods described herein can be used for screening of anti-human CD276 antibodies and in vivo drug efficacy testing, and can be used as a living substitute model for in vivo research, as well as screening, evaluation and treatment for human CD276 signaling pathway modulators.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric CD276.

2. The animal of claim 1, wherein the sequence encoding the human or chimeric CD276 is operably linked to an endogenous regulatory element at the endogenous CD276 gene locus in the at least one chromosome.

3. The animal of claim 1 or 2, wherein the sequence encoding a human or chimeric CD276 comprises a sequence encoding an amino acid sequence that is at least 70%, 75%, 80%, 85%, 90%, 95%, 99%, or 100% identical to human CD 276 (NP_001019907.1 (SEQ ID NO: 4)).

4. The animal of claim 1 or 2, wherein the sequence encoding a human or chimeric CD276 comprises a sequence encoding an amino acid sequence that is at least 70%, 75%, 80%, 85%, 90%, 95%, 99%, or 100% identical to SEQ ID NO: 13.

5. The animal of claim 1 or 2, wherein the sequence encoding a human or chimeric CD276 comprises a sequence that is at least 70%, 75%, 80%, 85%, 90%, 95%, 99%, or 100% identical to amino acids 34-417, 32-456, 30-459, or 26-459 of SEQ ID NO: 4.

6. The animal of any one of claims 1-5, wherein the animal is a mammal, e.g., a monkey, a rodent, or a mouse.

7. The animal of any one of claims 1-6, wherein the animal is a mouse.

8. The animal of any one of claims 1-7, wherein the animal does not express endogenous CD276.

9. The animal of any one of claims 1-8, wherein the animal has one or more cells expressing human or chimeric CD276.

10. The animal of any one of claims 1-9, wherein the animal has one or more cells expressing human or chimeric CD276, and a human CD276 receptor can bind to the expressed human or chimeric CD276.

11. The animal of any one of claims 1-9, wherein the animal has one or more cells expressing human or chimeric CD276, and an endogenous CD276 receptor can bind to the expressed human or chimeric CD276.

12. A genetically-modified, non-human animal, wherein the genome of the animal comprises a replacement of a sequence encoding a region of endogenous CD276 with a sequence encoding a corresponding region of human CD276 at an endogenous CD276 gene locus.

13. The animal of claim 12, where in the sequence encoding the corresponding region of human CD276 is operably linked to an endogenous regulatory element at the endogenous CD276 locus, and one or more cells of the animal expresses a chimeric CD276.

14. The animal of claim 12 or 13, wherein the animal does not express endogenous CD276.

15. The animal of any one of claims 12-14, where in the replaced sequence encodes all or a portion of the extracellular region (with or without signal peptide) of endogenous CD276.

16. The animal of any one of claims 12-15, wherein the animal has one or more cells expressing a chimeric CD276 having an extracellular region, a transmembrane region, and a cytoplasmic region, where in the extracellular region comprises a sequence that is at least 50%, 60%, 70%, 80%, 90%, 95%, or 99% identical to the extracellular region of human CD276.

17. The animal of claim 16, where in the extracellular region of the chimeric CD276 has a sequence that has at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 65 390, 400, 410, 420, or 430 contiguous amino acids that are identical to a contiguous sequence present in the extracellular region of human CD276.

18. The animal of any one of claims 12-17, where in the sequence encoding a region of endogenous CD276 comprises exon 2, exon 3, exon 4, exon 5, exon 6, exon 7, and/or exon 8, or a part thereof, of the endogenous CD276 gene.

19. The animal of claim 18, wherein the animal is a mouse, and the sequence encoding a region of endogenous CD276 starts within exon 3 and ends within exon 4 of the endogenous mouse CD276 gene.

20. The animal of any one of claims 12-19, where in the animal is heterozygous with respect to the replacement at the endogenous CD276 gene locus.

21. The animal of any one of claims 12-19, where in the animal is homozygous with respect to the replacement at the endogenous CD276 gene locus.

22. A method for making a genetically-modified, non-human animal, comprising: replacing in at least one cell of the animal, at an endogenous CD276 gene locus, a sequence encoding a region of an endogenous CD276 with a sequence encoding a corresponding region of human CD276.

23. The method of claim 22, where in the sequence encoding the corresponding region of human CD276 comprises exon 2, exon 3, exon 4, exon 5, exon 6, exon 7, exon 8, exon 9, and/or exon 10, or a part thereof, of a human CD276 gene.

24. The method of claim 22 or 23, wherein the sequence encoding the corresponding region of human CD276 starts within exon 3 and ends within exon 6 of a human CD276 gene.

25. The method of any one of claims 22-24, where in the sequence encoding the corresponding region of human CD276 encodes amino acids 34-417 or 32-456 of SEQ ID NO: 4.

26. The method of any one of claims 22-25, where in the region of an endogenous CD276 is located within the extracellular region.

27. The method of any one of claims 22-26, where in the sequence encoding a region of endogenous CD276 comprises exon 2, exon 3, exon 4, exon 5, exon 6, exon 7, and/or exon 8, or a part thereof, of the endogenous CD276 gene.

28. The method of claim 27, wherein the animal is a mouse, and the sequence encoding a region of an endogenous CD276 starts within exon 3 and ends within exon 4 of the endogenous mouse CD276 gene.

29. A non-human animal comprising at least one cell comprising a nucleotide sequence encoding a chimeric CD 276 polypeptide, where in the chimeric CD276 polypeptide comprises at least 50 contiguous amino acid residues that are identical to the corresponding contiguous amino acid sequence of a human CD276, where in the animal expresses the chimeric CD276.

30. The animal of claim 29, where in the chimeric CD276 polypeptide has at least 50, at least 100, at least 150, at least 200, or at least 250 contiguous amino acid residues that are identical to the corresponding contiguous amino acids sequence of a human CD276 extracellular region.

31. The animal of claim 29 or 30, where in the chimeric CD276 polypeptide comprises a sequence that is at least 90%, 95%, or 99% identical to amino acids 34-417, 32-456, 30-459, or 26-459 of SEQ ID NO: 4.

32. The animal of any one of claims 29-31, where in the nucleotide sequence is operably linked to an endogenous CD276 regulatory element of the animal.

33. The animal of any one of claims 29-32, where in the chimeric CD276 polypeptide comprises an endogenous CD276 transmembrane region and/or an endogenous CD276 cytoplasmic region.

34. The animal of any one of claims 29-33, where in the nucleotide sequence is integrated to an endogenous CD276 gene locus of the animal.

35. The animal of any one of claims 29-34, where in the chimeric CD276 has at least one mouse CD276 activity and/or at least one human CD276 activity.

36. A method of making a genetically-modified non-human animal cell that expresses a chimeric CD276, the method comprising: replacing at an endogenous CD276 gene locus, a nucleotide sequence encoding a region of endogenous CD276 with a nucleotide sequence encoding a corresponding region of human CD276, thereby generating a genetically-modified non-human animal cell that includes a nucleotide sequence that encodes the chimeric CD276, where in the non-human animal cell expresses the chimeric CD276.

37. The method of claim 36, wherein the animal is a mammal, e.g., a monkey, a rodent, or a mouse.

38. The method of claim 36 or 37, where in the chimeric CD276 comprises:
an extracellular region of human CD276, optionally comprising an endogenous signal peptide sequence; and
a transmembrane and/or a cytoplasmic region of endogenous CD276.

39. The method of any one of claims 36-38, where in the nucleotide sequence encoding the chimeric CD276 is operably linked to an endogenous CD276 regulatory region, e.g., promoter.

40. The animal of any one of claims 1-21 and 29-35, where in the animal further comprises a sequence encoding an additional human or chimeric protein.

41. The animal of claim 40, where in the additional human or chimeric protein is programmed cell death protein 1 (PD-1), IL4, Colony Stimulating Factor 1 (CSF1), IL34, C—C Motif Chemokine Receptor 2 (CCR2), CD40, C—X—C Motif Chemokine Receptor 4 (CXCR4), Vascular Endothelial Growth Factor (VEGF), cytotoxic T-lymphocyte-associated protein 4 (CTLA-4), Lymphocyte Activating 3 (LAG-3), B And T Lymphocyte Associated (BTLA), Programmed Cell Death Ligand 1 (PD-L1), CD27, CD28, CD47, CD137, CD154, T-Cell Immuno receptor With Ig And ITIM Domains (TIGIT), T-cell Immunoglobulin and Mucin-Domain Containing-3 (TIM-3), Glucocorticoid-Induced TNFR-Related Protein (GITR), Signal regulatory protein α(SIRPα) or TNF Receptor Superfamily Member 4 (OX40).

42. The animal of claim 40, wherein the additional human or chimeric protein is PD-1, and the animal expresses the human or chimeric PD-1.

43. The method of any one of claims 22-28 and 36-39, where in the animal further comprises a sequence encoding an additional human or chimeric protein.

44. The method of claim 43, where in the additional human or chimeric protein is PD-1, IL4, CSF1, IL34, CCR2, CD40, CXCR4, VEGF, CTLA-4, LAG-3, BTLA, PD-L1, CD27, CD28, CD47, CD137, CD154, TIGIT, TIM-3, GITR, SIRPα or OX40.

45. The method of claim 43, wherein the additional human or chimeric protein is PD-1, and the animal expresses the human or chimeric PD-1.

46. A method of determining effectiveness of an anti-CD276 antibody for the treatment of cancer, comprising:
administering the anti-CD276 antibody to the animal of any one of claims 1-21, 29-35, or 40-42, wherein the animal has a cancer; and
determining the inhibitory effects of the anti-CD276 antibody to the cancer.

47. The method of claim 46, where in the cancer comprises one or more cells that express CD276.

48. The method of claim 46 or 47, where in the cancer comprises one or more cancer cells that are injected into the animal.

49. The method of any one of claims 46-48, wherein determining the inhibitory effects of the anti-CD276 antibody to the cancer involves measuring the tumor volume in the animal.

50. The method of any one of claims 46-49, where in the cancer is liver cancer, pancreatic cancer, prostate cancer, osteosarcoma, breast cancer, colorectal cancer, stomach cancer, ovarian cancer, endometrial cancer, oral squamous cell carcinoma, cervical cancer, non-small cell lung cancer (NSCLC), bladder cancer, renal cancer, brain cancer, or melanoma.

51. A method of determining effectiveness of an anti-CD276 antibody and an additional therapeutic agent for the treatment of cancer, comprising administering the anti-CD276 antibody and the additional therapeutic agent to the animal of any one of claims 1-21, 29-35, 215 or 40-42, wherein the animal has a cancer; and
determining the inhibitory effects on the cancer.

52. The method of claim 51, where in the animal further comprises a sequence encoding a human or chimeric programmed cell death protein 1 (PD-1).

53. The method of claim 51 or 52, where in the animal further comprises a sequence encoding a human or chimeric programmed death-ligand 1 (PD-L1).

54. The method of any one of claims 51-53, where in the additional therapeutic agent is an anti-PD-1 antibody or an anti-PD-L1 antibody.

55. The method of claim any one of claims 51-54, where in the cancer comprises one or more cancer cells that express CD276, PD-L1, or PD-L2.

56. The method of any one of claims 51-55, where in the cancer is caused by injection of one or more cancer cells into the animal.

57. The method of any one of claims 51-56, wherein determining the inhibitory effects of the treatment involves measuring the tumor volume in the animal.

58. The method of any one of claims 51-57, where in the animal has liver cancer, pancreatic cancer, prostate cancer, osteosarcoma, breast cancer, colorectal cancer, stomach cancer, ovarian cancer, endometrial cancer, oral squamous cell carcinoma, cervical cancer, non-small cell lung cancer (NSCLC), bladder cancer, renal cancer, brain cancer, head and neck cancer, or melanoma.

59. A protein comprising an amino acid sequence, where in the amino acid sequence is one of the following:
(a) an amino acid sequence set forth in SEQ ID NO: 2, 4, or 13;
(b) an amino acid sequence that is at least 90% identical to SEQ ID NO: 2, 4, or 13;
(c) an amino acid sequence that is at least 91%, 92%, 93%, 94%, 95%, 96%, 97%, 98%, or 99% identical to SEQ ID NO: 2, 4, or 13;
(d) an amino acid sequence that is different from the amino acid sequence set forth in SEQ ID NO: 2, 4, or 13 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 2, 4, or 13.

60. A nucleic acid comprising a nucleotide sequence, where in the nucleotide sequence is one of the following:
(a) a sequence that encodes the protein of claim 59;
(b) SEQ ID NO: 1, 3, 5, 6, 7, 8, 9, 10, 11, or 12;
(c) a sequence that is at least 90% identical to SEQ ID NO: 1, 3, 5, 6, 7, 8, 9, 10, 11, or 12; and
(d) a sequence that is at least 91%, 92%, 93%, 94%, 95%, 96%, 97%, 98%, or 99% identical to SEQ ID NO: 1, 3, 5, 6, 7, 8, 9, 10, 11, or 12.

61. A cell comprising the protein of claim 59 and/or the nucleic acid of claim 60.

62. An animal comprising the protein of claim 59 and/or the nucleic acid of claim 60.


SEQUENCE LISTING (35 sequences)

SEQ ID NO: 1 - DNA, 3197 bp, Mus musculus
cggcgcggcgcgccaaagtgacctggtacagcctggaccccaagctcatcggctttgtctggctggccgcctggcctcttcccacttggatttggatgatcctgaggcctttggaggaacttcgagacaaaggcccctcttcctcttccacgggcaggagcagccattcgccacggagagcccagctgtcagctgtctcacaggaagatgcttcgaggatggggtggccccagtgtgggtgtgtgtgtgcgcacagcactgggggtgctgtgcctctgcctcacaggagctgtggaagtccaggtctctgaagaccccgtggtggccctggtggacacggatgccaccctacgctgctccttttccccagagcctggcttcagtctggcacagctcaacctcatctggcagctgacagacaccaaacagctggtgcacagcttcacggagggccgggaccaaggcagtgcctactccaaccgcacagcgctcttccctgacctgttggtgcaaggcaatgcgtccttgaggctgcagcgcgtccgagtaaccgacgagggcagctacacctgctttgtgagcatccaggactttgacagcgctgctgttagcctgcaggtggccgccccctactcgaagcccagcatgaccctggagcccaacaaggacctacgtccagggaacatggtgaccatcacgtgctctagctaccagggctatccggaggccgaggtgttctggaaggatggacagggagtgcccttgactggcaatgtgaccacatcccagatggccaacgagcggggcttgttcgatgttcacagcgtgctgagggtggtgctgggtgctaacggcacctacagctgcctggtacgcaacccggtgttgcagcaagatgctcacggctcagtcaccatcacagggcagcccctgacattcccccctgaggctctgtgggtaaccgtggggctctctgtctgtcttgtggtactactggtggccctggctttcgtgtgctggagaaagatcaagcagagctgcgaggaggagaatgcaggtgccgaggaccaggatggagatggagaaggatccaagacagctctacggcctctgaaaccctctgaaaacaaagaagatgacggacaagaaattgcttgattgggagctgctgcccttcccaggtggggggcccaccctctggcagtgttgagcttcaatgcgagcccttcccccaacgaatgggtttgtcccacagatctacccgttcgtcaaaggacgtggtccatagaccacccacagccttacttttccaatggacttaattcccatcatcctgcagcctcatttctccagtgacacgatacacgaaccatcctgcggccttatttcccacggacacgacacaaagatgtccctcctcggtgttcctccagagtcgtctggtggccttgtgatacggcgtgaaccttcttccttctgccttacgtctaatggacacacacgcaccacccccacacccttgctcctccaaagccatgcagactgtgtaactgctattattctccaaggggcatcctgtgcagatgaaaccctgctttatttccctgaagacagctgcacagtgacctcttagttcttgctcccatggccctgatgtatcctagttaccagccctcaacctcagttctgagggtgggatcccatcgctcagcaaggcttcatcctgacctccctgccctgatctgatctggccctggcttttgttgtctcgctccctgactaagtgagatggggcactctcccgcccccgccccccccaggtcacagatacctacctgcagctgtgcgtgctggatcacgcacatacttgccttgcatggtctcctggctgccctgggctgtgcctgttcttccataggaagcaagttcttgtctccctggttctcagggcccctcaggggctcagccttcagccctgtgcttccccatgttgggaatctttgttacctttttcttctttgtaaattaacatctgataacaaccacagggtccaatgggactttcacagacctgccagctagataaataatgacaacagaagtttattaatattttaagacttaggccttttgctgggcagcctcccaactattctatcctgactaatcctggcactatgtcccaccacatggccaggtctacctctctgctccactctccatccacctccatgtctgccagcaaatctcccgtgattcagttcttctcccagagtccctatctctgcccagaagtaccatcttcgacttcctgcccaactattggccgtcagctcttcattaaagccgatcagatgtaattctagattgccttaggcaggtgaggaagaaacaagtatttgtaaaatatgagaccagcaatgggccatagaaataacagcaccagatcctgccagcatttagccctctgttggtacaaaattaacaattgaatatacagagacctacttccagagtgtaccccaacaacaggcgtgagcatggtgctgggtactagggtcctgctggaaaatcagagaccttacctacagctgggacatgaccttgcttccgacttacccaccacttctggatacctcaccctcagcccacactatccctggcctagggcccagggtagagccagaaacatggagaaagcatggccccttgccgtacctggagaactgggtattttccagagtctttatagatgtggactggaaggcaggtggccacagccgtgcagacctgggtcaggtcagaaacctatgccatgctgggacctactcaacagcagaagcatgaagagggcctgaggacaagaaaggccttcttaccatggtgctattctggagctgggatatatacctggcttgtctctgactgccctggcttctggcagaacttctgatgtcctcctgaaggcctctctcccaccccagtacctgagaacctgaggataatttaaacatgggactctggccagcacctgggagagacaggtagatctctgatttttgactcagcctggtctatcgagtgagttccaggacatctggggctacacagagaaaccatcttaaagactaaaaataataaacatgagactgtaaactgggtgtattttgggagaaataaatgtctttttctttcaa

SEQ ID NO: 2 - protein, 316 aa, Mus musculus
MLRGWGGPSVGVCVRTALGVLCLCLTGAVEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSLQVAAPYSKPSMTLEPNKDLRPGNMVTITCSSYQGYPEAEVFWKDGQGVPLTGNVTTSQMANERGLFDVHSVLRVVLGANGTYSCLVRNPVLQQDAHGSVTITGQPLTFPPEALWVTVGLSVCLVVLLVALAFVCWRKIKQSCEEENAGAEDQDGDGEGSKTALRPLKPSENKEDDGQEIA

SEQ ID NO: 3 - DNA, 3295 bp, Homo sapiens
attcgggccgggcctcgctgcggcggcgactgagccaggctgggccgcgtccctgagtcccagagtcggcgcggcgcggcaggggcagccttccaccacggggagcccagctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctgaccatgaggaccagggagctgctacccctccctacagctcctaccctctggctgcaatggggctgcactgtgagccctgcccccaacagatgcatcctgctctgacaggtgggctccttctccaaaggatgcgatacacagaccactgtgcagccttatttctccaatggacatgattcccaagtcatcctgctgccttttttcttatagacacaatgaacagaccacccacaaccttagttctctaagtcatcctgcctgctgccttatttcacagtacatacatttcttagggacacagtacactgaccacatcaccaccctcttcttccagtgctgcgtggaccatctggctgccttttttctccaaaagatgcaatattcagactgactgaccccctgccttatttcaccaaagacacgatgcatagtcaccccggccttgtttctccaatggccgtgatacactagtgatcatgttcagccctgcttccacctgcatagaatcttttcttctcagacagggacagtgcggcctcaacatctcctggagtctagaagctgtttcctttcccctccttcctcctcttgctctagccttaatactggccttttccctccctgccccaagtgaagacagggcactctgcgcccaccacatgcacagctgtgcatggagacctgcaggtgcacgtgctggaacacgtgtggttcccccctggcccagcctcctctgcagtgcccctctcccctgcccatcctccccacggaagcatgtgctggtcacactggttctccaggggtctgtgatggggcccctgggggtcagcttctgtccctctgccttctcacctctttgttcctttcttttcatgtatccattcagttgatgtttattgagcaactacagatgtcagcactgtgttaggtgctgggggccctgcgtgggaagataaagttcctccctcaaggactccccatccagctgggagacagacaactaactacactgcaccctgcggtttgcagggggctcctgcctggctccctgctccacacctcctctgtggctcaaggcttcctggatacctcacccccatcccacccataattcttacccagagcatggggttggggcggaaacctggagagagggacatagcccctcgccacggctagagaatctggtggtgtccaaaatgtctgtccaggtgtgggcaggtgggcaggcaccaaggccctctggacctttcatagcagcagaaaaggcagagcctggggcagggcagggccaggaatgctttggggacaccgaggggactgccccccacccccaccatggtgctattctggggctggggcagtcttttcctggcttgcctctggccagctcctggcctctggtagagtgagacttcagacgttctgatgccttccggatgtcatctctccctgccccaggaatggaagatgtgaggacttctaatttaaatgtgggactcggagggattttgtaaactgggggtatattttggggaaaataaatgtctttgtaaaaa

SEQ ID NO: 4 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 5 - DNA, 2080 bp, Artificial Sequence
  nucleotide sequence of 73702275-73704354 of NCBI accession number NC_000015.10
cctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccggtgagcaccaggagggggacgccttccccttagttcaccctccattccccctgcagcccaccccctgctgcaccactgctcccagaaccccagtgctgattcctgtactcagccccatgcatccttttcgtcctctgccattgccctgcccttgacccctgccctctgtcacctccagctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggttgctttgcttaaatgtccccttgggggaggggggttctgcttctgtcggcaggggttgggagctccgaatctggccccaccttcaatctcccagaacagcaagttgcctcctaatgatagggagaaagataacggggaggtggggatgttcactggaggggttgggggcggagaaatcacaggccttcttaaggctctcctgacttcaaggctctggaagcctctgggaagaatggagccacatctgggtcagcagaggggtgggcgaaggaaaggagtcctgatggcaggattcaccagggctggagctggtgagagtcacctgccccaggcctggccctgggattggtctggtcaggccaagacccacctgctgtgattgttctcagaagattggggagactccatatctgagagtcctcagacttaatctgcttttgcatctaagaagaaaataggaagatggaacagggtctgggaattgatgggggaagagtttgggggactcgggtggtagcctagagagtcccatttctcctcaccaccctgcccctctgaccccgcccccaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccggtgagcaccaggagggcgacgccttccccttggttcaccctccattccctctgcagcccaccctctgctgcaccactgctcccagaaccccagtgctgattcctgtactcagccccatgcatccttttcctcccctgccattgccctgcccttgacccctgccctctgtcacctccagctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcag

SEQ ID NO: 6 - DNA, 4063 bp, Artificial Sequence
  5′ homologous arm
tctatctctggaggtggtcatggatacaaaatgttcatattcttgctaattactgttaggtgggacgggtggacatgggtcacatggcttgagcaggcaacagcaagagacacaagaagtgcctgcagcctgcctgagtcaggcacctgcaagctaaacccaggggagtctgggaccaccctgggctaggcttggtctgttgtaagcgatccaactcttgctgaggcctgacgctgaccccagccctgcccctggtctctttcttctctgctgggccttggttgtttggtacatgtgtattaaactagcagatgtctctcccagactccacatccttacatttctgctactcctagctagggctctgtcctggggtttctgcacacatgctcctaacctaacatgccttggacaaaaggctccacatttcctgccccaataaccaggtctcagaaccaagtgtcttgacagttgccttcccgtgtcctatgctctcagatactgtcactgtgatctcttcctgccttatcactcggctggcccctttcatctaccctgcacatgctatcgaagtagccctgacaatgtggtttctttattccagaaccctcagtggctcctggctacctttagtagttgagtagacgtttcaagttgaaccctaagtctgtctaaagtctacccctttactgtcccatttctcccatcagccaaagtgacctggtacagcctggaccccaagctcatcggctttgtctggctggccgcctggcctcttcccacttggatttggatgatcctgaggcctttggaggaacttcgagacaaaggcccctcttcctcttccacgggcaggagcagccattcgccacggagagcccagctgtcagctgtctcacaggaagatgcttcgaggatggggtggccccagtgtgggtgtgtgtgtgcgcacagcactgggggtgctgtgcctctgcctcacaggtgaggggaagggtcatgatcatgatggtatgtggggatgtccactcctccctgggctccagccaggctgtctctgtcttgaagaagcatattgatttccatctctgggctccacaggccaccaacctcctttaccttagcctctgcttcttcatctgtaaaatgggacagtatgctatgataatgcaagttcctgaacacactgtgtgaactctgctcaggaccattgccgaaaactttctgtcctcagacacaggcatgactccattctttattttgttgcaaacctttgctcagatgacaggtcatcagcatgctttcttgactactttgtataaaacaacatagcactcttcctccctccctaaatcatccctgccccgagcgctttattacattcttcctataatttacctccactggacatatggttcctttgcttattgcctgttgtcacctcgctcataaggcaaggtctaacctcagggactgaatggatgattgctatgttcacagtgctctgtccccatgctgaataaattcccacctagtgcttggctaagtaaatgccctgtttaggtagtcatccaccttatggagaaaacaaagccaagtgtctggcgccatctagtggtcatcagagacaacaccatctctatccgtgggtgttcagacttctttttgtgtgagtgtgtttgcttggttgagacagggtctcactatgtgtacctggctgtccttgaactcattatctagacaggcaggcctcaaactcacacagagatctacctgtctctgcctcccaagtgttgggataaaaggtgtgtgtcaccacacttggcctggttctaacttctttagaaagaattttgggtgactaagctccatgtcctagatgtcccctacaaggggacacgtagatctagttggcaaagatgaaaactggcctgagttttcatggaatcactgaaagatatcaagggccagcttcaacctctagacatcacatactaagaaagagtgtgtgtagaattcatagtaaaagactaatcacaaaacaaatacaaattttctaagccttatatgggtgctgtattcagctggcttcatttgaagcttgtttgtgctggaaagtttataggatagaaggcacgggggccaatagcggagcctgtttctttctctctctctctctcccttcctttctccctccctccctccctccctccctccctccctccctccctccctccctcccccctctctttctttctttctttctttctttctttctttctttctttctttctttctttctttctttctttctttcttttttttttgttttttgtttttttgagtcagggtttctctgtataaccctggctgtcctgaaactcactctgtagaccaggctggcctcgaactcagaaatccacctgcctctgtctcccaagtgctgggattaaaggcgtgcgccaccactgcccggcaccattttcttataacttataaaagaaagcatttcctttgggggcttgcttaacaatgtcagagggtgagtccatgaccctctccattatggtggagaatatgacagcaggcaggcccgcatagcactggagcagtagctgagagattccatccgatccacaagcacaaagcagagaggagagcacaggcttttgaaacctcaaagcccatgctagttacagatctcctctaacaaggccacacctcctaatccttcccaaacagttccaacagcattcaaacatatgcacgcgaagggggggggggcacattctcatttaaatcaccacacattccttccttctcacccttcatcaaccagcaaatattgataattctctgggctgcatcttagatctggctggtttgctcaatccctgtggctgctcttcactgcagggcctagaacttgctatgttgaccaggctggcttcaaatttatggagatcttcctgcctctgcctcctgagtgcttagcctttcgctgacttgtgctcatagcctcatgtgtcctttctacagctgtgtcgccatcacttcttttgaggacctctgagctggtctcccgactacccaccccctcactcttccacgatagagtctccctttatagacgagccggggtgagcttcaggctttgctacccagatcacatctaataattagtgccctccgtaatgacatcctctcgaaggtgggagaccccaggtttcttccagcatctctcagagccccgtctggctggcctccagtgggcttttccatgtgctctttctgatctgcacctcagggcctttgtacctgctggtccctcttcctgggacacacttacatgtgtttcatagagctcctgcctcacaccttatttaaagacatgggtattctttcttgcatagaagttaagtatgtttattcatgaagagaagagaaggaaaggcactcatgaagttctctcacccaagggagttcactgagaacagtgggtgtttagttctgcaggcacccttcctatacagatagtatttgtaggaaaatgcaagggacctggcaggtggaaagaagctgccaggcttgaaagctgcctcttctttggttctttctttttctttgacttttataagtacttcctctttcttctccacccacagcttcccttttttttttttttttttttttttttagaatagagcggatccaaagctgggtgatgctctaacaccaagccctccccctcccccacattgtctaatcacaagctgatgaacatctgtgtccatcacaaactgcctgccttccattccgacataaacgagctccagggtatgacctgggtcttggtccttgttgcatttctaattcatgttggcaaatagctgcctgcagtgaggaacatagataggaacttactctgagcctctgacctgaaaggaagcgtgtgcagagcctagaggtagctggaaaaggagagagaccctgaggaaacctcatctttcctaactgccctaccccttccaactcacccccaggagctgtggaagtccaggtc

SEQ ID NO: 7 - DNA, 5208 bp, Artificial Sequence
  3′ homologous arm
cttcagtgcttggggatctaaagagggttcaggctcaccttcatgccaaggtggacagtcactctctgtgtttggccttgacatgtctctgttcagtcctttagtagatctgtgtgctgggtctagcttcccagtgcctccctctgggctgtgtgagctttcccagggtcctctctgtctctgagatcagggtgtctgtcactggtccttatcttttctcatcccataacttctgcctggcaccctcagcccacccagctttctacttccaagggttgccagtgtctgtgtagaccttaggctcattgcggctcacctctcctgctgagcgtgctggaccttgtgtgggtttctctcctgatgaactcagccagtggccagcattcatgcttatctcccaggagtaagtcttattcctaactcttgcttttttaaggtgtcacctagacctccccaagactgaacctaccttcgctgtagagagccagtctatgcagcctctaagggagtcccttcctcagctctctcatattctgaggtaccaccttgatcttacagagaacatcgtaggaaagccgtgtttcactgggttggagagatgccttagtgactaaaaagccttgatgctctatcattgaagaccacagagtttacatccaagcacacatatatcacgtggcttgcaaacatctataattccaactccaagggatcagatgctctttgctggcctctgtggataccctcacacacatgtgtgaacacacgcacgtgtgcgtgcacacacacacacacacacacacacacgcatgaacattcttttttaaaaaacaacaacaacaacaacaacaggtctgtttttttttttgttgttgttgttgtttttgtttttataagaaagaccaaggagtctggagtgagatgtaagatggacagatgtgtggaggttatcaggaaggacagaggactcatgatcaacctgtaccaagtgggctgggatgtctgtagtatgcttgctgtgaagcaagggtttgtatgtggaaagaacagttgccctgcttcagagaacattacctcagtagatagcattgactgaaagggatttcatttgggctgttgtcgtcaagcacaattcagacgagatggaggctcagggaaaagggactctctcccaagaccacagaccagcacagtggctccaaaggatttgggggccgggccccactttgaaaactgacaagtgctaaaggcagtggtttgcctgaagtttggcgggttcccagcttctctaaactccatctgaggagcctttggatccttttgcggcattctgcagcgagcagaggagccaggatctgaatctagcattggcctcttagcccagtttctctcctccccaccggctggcctcctgtttccattagagcagagctggcttaccttggagcccttggcctgcatttggctgcgggcagctctgccttgtacagcattctgggatttcagtgctcagtcctctgcctggtctccctccacctgaagggagttagtgatgttgagtggcctgtgccaggcatctctggagagagagggctttctagagcaaacactggaaatgtaggtctgagccccactaactgtgtccttggccaggaggatgaaggcagtctcaccctgctttaggctccgtgtcagggtgaaccttacctttgagtatttcagttttttattagcttctattaattacatatcaaatagattatatttcatatatatatgaaatataatatataatatatacatacattctgatcatgctcaccccaacaccatttcatgtcccctgtcattccatctgatccccttcctctgcccaactagtctcctccgattttcattactctaaaaccttagagtgtgtgtgtgcgcgcgtgcgtgcgtgcgcgtgtatgtgtgtgaaggtcagaggacaactttcaggaattggtgctctccttccaccattggttctggggctagaactcaggtcaccaggcattcagagtagactcttttaccagttggtgtccttctcccatcttaatgtctctatatattttgtgacactatggcacagtgaggggtcagttataggagcttgggcactttatcaagtgaccatgtttccttccccctagcgaccattagttgcctgcagttcttcagggagggtggagtcccttgggtatttctgctgctctgccctgctcactgctgcaggagcaccttagacatcttagacatttccatgtgagggtttcacatgggctctagactgacctagtcacccacctcagctgtggcctctgctcctgccctggcttcttctgcttcagactccagagcagcagtgctccacccccctgctcccaccccatgtctgtgcaaagcatagctactgccaagacttaccacaattcctggatgcaggctgccgggtcccgctgcctgggttctccactgtggctgctgatgccctggctgcctgctgtttccagagagcataaggaatcaagctgtctttgttagtgccccaacctgcaccggcagcggctctgccctggacccagtgtggcactgggctcaggatgatagctatgctggagtcatgtggggtatgtgctcagaacagctgctggtcccaatcccaccttagttgctctgcctccacacactccttgacatgagatatctaatggggagcctcacgttgcacgtgaatattggcgaggctctcccagatctgtatggatccttaagtgacttagactctcactccaggccctcatctatgtgcagtgagtgacagagagtactttgtgggcggtgttccatctctctgctgatgattgattggcaggatctccttgtcgcagttttactgttgattgattgattgactggcaggatctcagttgaccttgaactaaatatgtcacgaggatgaccttgagtttcttctcctcccacctctacctccttagttcttggattccagggggctcgggaaacaatgcccagttcatgtgctgctgggaatagaacccggagcttcttgtgttttaggcaagcaaatggtctaccaactgagatacatctccagagaaccatagctttcctttccgttcttttttttctttctcctttctttttgtagcccctttccaaatataaaagctaatactagctcctggattgtccaaagcaggccacagtctgactcatgggctgtcgctggctgatgcctgatcttcaggatgatgtaaaccaggtaggactgtaacctcgagggaggtgtctggagaggtgattgctaggaagggaaaatgacagcgactgtgtagatcctggaaggagtcaggactaattctgtagagaatggccgaagtggggtttcctgtggcgacattagacagaaaagctgggagctgggtcagcttagagacccttagagatccagccattcgtgcctgcgtgtcttggggcacatcactgttttagtttagtttatttggtgcagctgtaagaggattcctgagacaggtcatgtaaagaacagacatttggtgatagctcagtggttaagagcactggctgcccttccagaggtcctgagttcaattcccagcaactacatgatggcttacaaccatatcagaatgaaatctgatgccccctgtctggtatgccggtgtacatacagatatacattaaataaataaaacttaaaaaaaaaaaagaaagaacatttactggcatgtgcatctcaagctgagagtaccatacccagacagctgggagagcagagaaagaaagctcttgccacagaaggatgaaggctggtgtttggaatccccctcttagaagacagaacggttatcctaggtcagtgaggccctatccatggttattagtcagcctcaggcttcttagcttcccctcttcccttctgaggtgaacccatagccacaatggcagggtttggtggggtgagtagagctgtgactctgggagccctggccatgggagcctcagtgagcatcattgaccttggtggcgtaggagcttgggagcccctgttcacaccgtagaacaagatggtctcatgcttctttcttcctcttctagggcagcccctgacattcccccctgaggctctgtgggtaaccgtggggctctctgtctgtcttgtggtactactggtggccctggctttcgtgtgctggagaaagatcaagcagagctgcgaggaggagaatgcaggtgagggagagtgactcgcgtgtgcgtgcgtgcgtgtgtttgtgtgtatgtgtgtgtgtgtgtgcgagtgttgtacatacatgcctgcatgtcttggggcatgtcactgttttcgttttttggttcagctgtaagagaatacccgagacagggtcatgtaaaggacagacatgtactggcttgtgtttctcaagctgtgggtatcctatccagacagctgggagagcacagaaaacacttgccacagaaggacgaaagttggagtttgcaaccctagcacctacataaatgtctagtgggcatggcagcttgtctgtaattccagctcttagaaggaggagacatgagcttccccagccagactagccacatcagtgagctctgggttcaactgagagacctggtcttaatgaataagcatggagtgaccaagaagactcctgacatcacccacacacatgtaaacaggcatgcacacatgcacgagtaccacacactcggctgcatgcacaaagtaagtaagtcaataccatgtccaggatctgcccccctggagaggccttgctgctcattaaatggtggaaagcgagagggcaaaggccatagcgcccatcctatctgcaagggtggggtggtcagagcctaattaatcaccctttagaagcatcgcctctttaggaaacaaaattaatgtaaaagttaaatttatttttttaatttaaagtttttgtatatgggtgttttgcctgcatgcaggtctggaactgaagtcacagtggttattagctgccacatggctgctgagaacagaatcttgttctctataagagcagacagtgctctttaaccactgagccatcttctcctgcccagaagtcacaccccttgatactgtttgatggcagc

SEQ ID NO: 8 - DNA, 61 bp, Artificial Sequence
  the connection between the 5′ end of the human CD276 gene sequence in the A fragment and the mouse CD276 gene locus
tcacccccaggagctgtggaagtccaggtccctgaagacccagtggtggcactggtgggca

SEQ ID NO: 9 - DNA, 71 bp, Artificial Sequence
  the connection between the 3′ end of the human CD276 gene sequence and the mouse CD276 gene locus
actggcaacgtgaccacgtcgcagatggccaacgagcagggcttgttcgatgttcacagcgtgctgagggt

SEQ ID NO: 10 - DNA, 104 bp, Artificial Sequence
  the connection between the 5′ end of the Neo cassette and the mouse CD276 gene locus
cttaaatctctggtttccaggtcagcactagttcaagtaggcaccaattgaagcttgatatcgaattccgaagttcctattctctagaaagtataggaacttca

SEQ ID NO: 11 - DNA, 100 bp, Artificial Sequence
  the connection between the 3′ end of the Neo cassette with the mouse CD276 sequence
aagttcctattctctagaaagtataggaacttcatcagtcaggtacataatggtggatcccttcagtgcttggggatctaaagagggttcaggctcacct

SEQ ID NO: 12 - DNA, 3851 bp, Artificial Sequence
  mRNA sequence of the engineered mouse CD276 after humanization
cggcgcggcgcgccaaagtgacctggtacagcctggaccccaagctcatcggctttgtctggctggccgcctggcctcttcccacttggatttggatgatcctgaggcctttggaggaacttcgagacaaaggcccctcttcctcttccacgggcaggagcagccattcgccacggagagcccagctgtcagctgtctcacaggaagatgcttcgaggatggggtggccccagtgtgggtgtgtgtgtgcgcacagcactgggggtgctgtgcctctgcctcacaggagctgtggaagtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgttcgatgttcacagcgtgctgagggtggtgctgggtgctaacggcacctacagctgcctggtacgcaacccggtgttgcagcaagatgctcacggctcagtcaccatcacagggcagcccctgacattcccccctgaggctctgtgggtaaccgtggggctctctgtctgtcttgtggtactactggtggccctggctttcgtgtgctggagaaagatcaagcagagctgcgaggaggagaatgcaggtgccgaggaccaggatggagatggagaaggatccaagacagctctacggcctctgaaaccctctgaaaacaaagaagatgacggacaagaaattgcttgattgggagctgctgcccttcccaggtggggggcccaccctctggcagtgttgagcttcaatgcgagcccttcccccaacgaatgggtttgtcccacagatctacccgttcgtcaaaggacgtggtccatagaccacccacagccttacttttccaatggacttaattcccatcatcctgcagcctcatttctccagtgacacgatacacgaaccatcctgcggccttatttcccacggacacgacacaaagatgtccctcctcggtgttcctccagagtcgtctggtggccttgtgatacggcgtgaaccttcttccttctgccttacgtctaatggacacacacgcaccacccccacacccttgctcctccaaagccatgcagactgtgtaactgctattattctccaaggggcatcctgtgcagatgaaaccctgctttatttccctgaagacagctgcacagtgacctcttagttcttgctcccatggccctgatgtatcctagttaccagccctcaacctcagttctgagggtgggatcccatcgctcagcaaggcttcatcctgacctccctgccctgatctgatctggccctggcttttgttgtctcgctccctgactaagtgagatggggcactctcccgcccccgccccccccaggtcacagatacctacctgcagctgtgcgtgctggatcacgcacatacttgccttgcatggtctcctggctgccctgggctgtgcctgttcttccataggaagcaagttcttgtctccctggttctcagggcccctcaggggctcagccttcagccctgtgcttccccatgttgggaatctttgttacctttttcttctttgtaaattaacatctgataacaaccacagggtccaatgggactttcacagacctgccagctagataaataatgacaacagaagtttattaatattttaagacttaggccttttgctgggcagcctcccaactattctatcctgactaatcctggcactatgtcccaccacatggccaggtctacctctctgctccactctccatccacctccatgtctgccagcaaatctcccgtgattcagttcttctcccagagtccctatctctgcccagaagtaccatcttcgacttcctgcccaactattggccgtcagctcttcattaaagccgatcagatgtaattctagattgccttaggcaggtgaggaagaaacaagtatttgtaaaatatgagaccagcaatgggccatagaaataacagcaccagatcctgccagcatttagccctctgttggtacaaaattaacaattgaatatacagagacctacttccagagtgtaccccaacaacaggcgtgagcatggtgctgggtactagggtcctgctggaaaatcagagaccttacctacagctgggacatgaccttgcttccgacttacccaccacttctggatacctcaccctcagcccacactatccctggcctagggcccagggtagagccagaaacatggagaaagcatggccccttgccgtacctggagaactgggtattttccagagtctttatagatgtggactggaaggcaggtggccacagccgtgcagacctgggtcaggtcagaaacctatgccatgctgggacctactcaacagcagaagcatgaagagggcctgaggacaagaaaggccttcttaccatggtgctattctggagctgggatatatacctggcttgtctctgactgccctggcttctggcagaacttctgatgtcctcctgaaggcctctctcccaccccagtacctgagaacctgaggataatttaaacatgggactctggccagcacctgggagagacaggtagatctctgatttttgactcagcctggtctatcgagtgagttccaggacatctggggctacacagagaaaccatcttaaagactaaaaataataaacatgagactgtaaactgggtgtattttgggagaaataaatgtctttttctttcaa

SEQ ID NO: 13 - protein, 534 aa, Artificial Sequence
  encoded protein sequence of the engineered mouse CD276 after humanization
MLRGWGGPSVGVCVRTALGVLCLCLTGAVEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPLTFPPEALWVTVGLSVCLVVLLVALAFVCWRKIKQSCEEENAGAEDQDGDGEGSKTALRPLKPSENKEDDGQEIA

SEQ ID NO: 14 - DNA, 20 bp, Artificial Sequence
  primer
gctcgactagagcttgcgga

SEQ ID NO: 15 - DNA, 25 bp, Artificial Sequence
  primer
tctgttcatcctctcacaggcacag

SEQ ID NO: 16 - DNA, 26 bp, Artificial Sequence
  primer
ctgtgtacctgaggctagaccacatg

SEQ ID NO: 17 - DNA, 20 bp, Artificial Sequence
  primer
agggatgcgttgccctgtgc

SEQ ID NO: 18 - DNA, 25 bp, Artificial Sequence
  probe
ggcagcagatggggctaagatgaat

SEQ ID NO: 19 - DNA, 26 bp, Artificial Sequence
  probe
cgaaagactacagagtgctttggcct

SEQ ID NO: 20 - DNA, 25 bp, Artificial Sequence
  probe
tgcgtttgggagggaaacagacatt

SEQ ID NO: 21 - DNA, 25 bp, Artificial Sequence
  probe
aactgtgaggctctgaacattcggc

SEQ ID NO: 22 - DNA, 21 bp, Artificial Sequence
  probe
ggatcggccattgaacaagat

SEQ ID NO: 23 - DNA, 22 bp, Artificial Sequence
  probe
cagaagaactcgtcaagaaggc

SEQ ID NO: 24 - DNA, 25 bp, Artificial Sequence
  primer
actctgagcctctgacctgaaagga

SEQ ID NO: 25 - DNA, 22 bp, Artificial Sequence
  primer
gaagcagagggtacccctctcc

SEQ ID NO: 26 - DNA, 23 bp, Artificial Sequence
  primer
tgtagaccatgtagttgaggtca

SEQ ID NO: 27 - DNA, 20 bp, Artificial Sequence
  primer
agggatgcgttgccctgtgc

SEQ ID NO: 28 - DNA, 24 bp, Artificial Sequence
  primer
agactcacactggaggagtctctt

SEQ ID NO: 29 - DNA, 23 bp, Artificial Sequence
  primer
tcagagacagagaggaccctggg

SEQ ID NO: 30 - DNA, 20 bp, Artificial Sequence
  primer
ggaccaaggcagtgcctact

SEQ ID NO: 31 - DNA, 23 bp, Artificial Sequence
  primer
tggtcaccatgttccctggacgt

SEQ ID NO: 32 - DNA, 21 bp, Artificial Sequence
  primer
cagctggtgcacagctttgct

SEQ ID NO: 33 - DNA, 23 bp, Artificial Sequence
  primer
ggcagctgtaggtgccatttgca

SEQ ID NO: 34 - DNA, 21 bp, Artificial Sequence
  primer
aggtcggtgtgaacggatttg

SEQ ID NO: 35 - protein, 316 aa, Rattus norvegicus
MLRGWGGPSVGVSMGTALGVLCLCLTGAVEVQVSEDPVVALVDTDATLRCSFSPEPGFSLRQLNLIWQLTDTKQLVHSFTEGRDQGSAYANRTALFPDLLVQGNASLRLQRVRVTDEGSYTCFVSIQDFDSAAVSLQVAAPYSKPSMTLEPNKDLRPGDMVTITCSSYQGYPEAEVFWKDGQGLPLTGNVTTSQMANERGLFDVHSVLRVVLGANGTYSCLVRNPVLQQDAHGSVTITGQPMTFPPEALWVTVGLSVCLVILLVALAFVCWRKIKQSCEEENAGAEDQDGDGEGSKTALRPLKHSENKEDDGQEIA